(19) United States
(12) Patent Application Publication
(10) Pub. No.: US 2005/0119252 A1
(43) Pub. Date: Jun. 2, 2005

(54) Ligands of melanocortin receptors and compositions and methods related thereto

(75) Inventors: Fabio C. Tucci, San Diego, CA (US); Joe Anh Tran, San Marcos, CA (US); Wei-Chuan C. (Caroline) Chen, San Diego, CA (US); Wanlong Jiang, San Diego, CA (US); Chen Chen, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA (US)

(21) Appl. No.: 10/971,732

(22) Filed: Oct. 22, 2004

Related U.S. Application Data
(60) Provisional application No. 60/513,727, filed on Oct. 22, 2003.

(51) Int. Cl. A61K 31/506; A61K 31/551; C07D 417/14; C07D 413/14; C07D 43/14

(52) U.S. Cl. 514/218; 514/252.18; 514/252.19; 544/295; 540/575

(57) ABSTRACT

Compounds which function as melanocortin receptor ligands and having utility in the treatment of melanocortin receptor-based disorders. The compounds have the following structure (I): 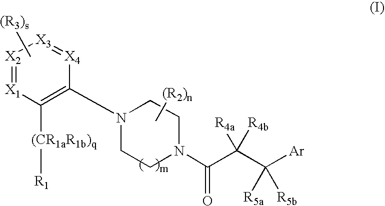
including stereoisomers, prodrugs, and pharmaceutically acceptable salts thereof, wherein m, n, q, s, R₁, R₁ₐ, R_{1b}, R₂, R₃, R₄ₐ, R_{4b}, R₅ₐ, R_{5b}, X₁, X₂, X₃, X₄ and Ar are as defined herein. Pharmaceutical compositions containing a compound of structure (I), as well as methods relating to the use thereof, are also disclosed.

### CROSS-REFERENCE TO RELATED APPLICATION

This application claims priority to U.S. Provisional Application Ser. No. 60/513,727, filed Oct. 22, 2003, the entire disclosure of which is incorporated by reference herein.

### BACKGROUND OF THE INVENTION

1. Field of the Invention

This invention is generally directed to ligands of a melanocortin receptor, as well as to compositions and methods for using such ligands to alter activity of a melanocortin receptor.

2. Description of the Prior Art

Melanocortin (MC) receptors are members of the family of G-protein coupled receptors. To date, five distinct MC receptors (i.e., MC1-R, MC2-R, MC3-R, MC4-R and MC5-R) have been identified in a variety of tissues and these receptors have been shown to mediate a number of physiological processes. Ligands, including peptides and small molecules, have been shown to act as agonists or antagonists at these receptors.

The role of specific MC receptors in physiological processes has been the object of intense study since their discovery and cloning. These receptors are expressed in a variety of tissues including melanocytes, adrenal cortex, brain, gut, placenta, skeletal muscle, lung, spleen, thymus, bone marrow, pituitary, gonads and adipose tissue. A putative role of MC receptors has been shown in melanocytes, stimulatory actions on learning, attention and memory, motor effects, modification of sexual behavior, facilitation of nerve regeneration, anti-inflammatory and antipyretic effects, and the regulation of food intake and body weight.

The pro-opiomelanocortin (POMC) gene product is processed to produce a number of biologically active peptides that are expressed in the pituitary, and two locations in the brain: the arcuate nucleus of the hypothalamus and the solitary tract nucleus of the brain stem. These peptides elicit a range of biological activities. Two POMC peptides, α-melanocyte stimulating hormone (α-MSH) and adrenocorticotropic hormone (ACTH), control melanocyte and adrenocortical function, respectively, in the periphery.

Cloning studies have defined a family of five melanocortin (MC) receptors that respond to POMC peptides (reviewed in Rec. Prog. Hor. Res. 51:287-318, 1996). Each receptor in this family is pharmacologically distinct in its particular response to the POMC peptides α-MSH, γ-MSH and ACTH and to two peptide antagonists. MC4-R differs from the other MC receptors in that it binds both natural melanocortin antagonists, agouti (Nature 371:799-802, 1994) and agouti-related protein (AgRP) (Biochem. Biophys. Res. Commun. 237:629-631, 1997). In contrast, MC1-R only binds agouti, MC2-R does not bind AgRP, MC3-R only binds AgRP, and MC5-R has only low affinity binding for AgRP (Mol. Endocrinology 13:148-155, 1999).

The expression of specific MC receptors is restricted anatomically. MC1-R is expressed primarily in melanocytes, while MC2-R is expressed in adrenocortical cells. MC3-R is expressed in brain, placenta and gut, and MC4-R is expressed primarily in the brain where its mRNA can be detected in nuclei that bind α-MSH. MC4-R is notably absent from adrenal cortex, melanocyte and placental tissues. Both MC3-R and MC4-R are expressed in arcuate and paraventricular neurons. MC5-R is expressed in brain, adipose tissues, muscle and exocrine glands.

α-Melanocyte stimulating hormone (α-MSH) is a tridecapeptide whose principal action (i.e., the activation of a set of G-protein coupled melanocortin receptors), results in a range of physiological responses including pigmentation, sebum production and feeding behavior. Cyclized peptide derivatives of α-MSH are potent modulators of these receptors. When administered by intracerebroventricular (i.c.v) injection into fasted animals, peptides exhibiting MCR-4 antagonist activity increase food intake and body weight. Moreover, overexpression of a naturally occurring peptide antagonist, agouti-related peptide (AgRP) has a similar effect on food intake and body weight. The development of small molecule antagonists of the MC4-R would selectively enhance the feeding response. MC4-R antagonists have a unique clinical potential because such compounds would stimulate appetite as well as decrease metabolic rate. Additionally, chronic MC4-R blockade causes an increase in lean body mass as well as fat mass, and the increase in lean body mass is independent of the increase in fat mass. Orally active forms of a small molecule MC4-R antagonist would provide a therapeutic strategy for indications in which cachexia is a symptom.

The MC receptors are also key mediators of steroid production in response to stress (MC2-R), regulation of weight homeostasis (MC4-R), and regulation of hair and skin pigmentation (MC1-R). They may have additional applications in controlling both insulin regulation (MC4-R) and regulation of exocrine gland function (MC5-R) (Cell 91:789-798, 1997); the latter having potential applications in the treatment of disorders such as acne, dry eye syndrome and blepharitis. Melanocortin peptides have also been reported to have anti-inflammatory activity, although the receptor(s) involved in mediating these effects have not yet been determined. Endocrine disorders such as Cushing's disease and congenital adrenal hyperplasia, which are characterized by elevated levels of ACTH, could be effectively treated with ACTH receptor (MC2-R) antagonists. Some evidence suggests that depression, which is characterized by elevated levels of glucocorticoids, may also be responsive to these same compounds. Similarly, elevated glucocorticoids can be an etiological factor in obesity. Synthetic melanocortin receptor agonists have been shown to initiate erections in men (J. Urol. 160:389-393, 1998). An appropriate MC receptor agonist could be an effective treatment for certain sexual disorders.

MC1-R provides an ideal target for developing drugs that alter skin pigmentation. MC1-R expression is localized to melanocytes where it regulates eumelanin pigment synthesis. Two small clinical trials indicate that broad-spectrum melanocortin agonists induce pigmentation with limited side effects. The desired compound would have a short half-life and be topically applied. Applications include skin cancer prevention, UV-free tanning, inhibition of tanning and treatment of pigmentation disorders, such as tyrosinase-positive albinism.

The role of melanocortin receptors in regulation of adiposity signaling and food intake has been recently reviewed (Nature 404:661-669, 2000). Direct experimental evidence for the individual role of MC4 and MC3 receptors in energy homeostasis has not yet been reported due to the lack of potent and specific MC4 and MC3 agonists. Central administration of synthetic, non-selective MC-3R and MC4-R agonists, such as cyclic side-chain-lactam-modified peptide MT-II suppresses food intake in rodents and monkeys, and stimulates energy expenditure resulting in reduced adiposity (Endocrinology 142:2586-2592,2001). Conversely, selective peptide antagonists of the MC4 receptor stimulate food consumption and result in increased body weight, suggesting the main effects of agonist induced inhibition of food consumption are mediated by MC4-R receptor activity. (European J. Pharmacol. 405:25-32, 2000). Selective small molecule MC4-R antagonists also stimulate food intake in animal models of cachexia.

Genetically modified animals lacking the MC4-R receptor are hyperphagic and obese (Cell 88:131-141, 1997). Humans with defective melanocortin 4 receptors exhibit marked hyperphagia and increased body mass relative to their normal siblings (Nature Genet. 20:111-114, 1998). In addition, studies with mice lacking functional MC-3 receptors suggest that agonist stimulation of this receptor may also play a role in control of energy homeostasis, feeding efficiency, metabolism and bodyweight (Endocrinology 141:3518-3521, 2000). Therefore MC4-R and MC3-R agonists may be useful in the control of obesity and in treatment of related disorders including diabetes.

Due to their important biological role, a number of agonists and antagonists of the MC receptors have been suggested. For example, U.S. Pat. No. 6,054,556 is directed to a family of cyclic heptapeptides which act as antagonists for MC1, MC3, MC4 and MC5 receptors; U.S. Pat. No. 6,127,381 is directed to isoquinoline compounds which act upon MC receptors for controlling cytokine-regulated physiologic processes and pathologies; and published PCT Application No. WO 00/74679 is directed to substituted piperidine compounds that act as selective agonists of MC4-R. Published PCT Application No. WO01/05401 is directed to small peptides that are MC3-R specific agonists. Recent PCT publications WO02/059095, WO02/059107, WO02/059108, WO02/059117, WO03/009847 and WO03/009850 describe melanocortin receptor agonists which may be useful for the treatment of obesity, among other diseases. WO03/031410 and WO03/068738 describe certain compounds which act at melanocortin receptor(s).

Accordingly, while significant advances have been made in this field, there is still a need in the art for ligands to the MC receptors and, more specifically, to agonists and/or antagonists to such receptors, particularly small molecules. There is also a need for pharmaceutical compositions containing the same, as well as methods relating to the use thereof to treat conditions associated with the MC receptors. The present invention fulfills these needs, and provides other related advantages.

### BRIEF SUMMARY OF THE INVENTION

In brief, this invention is generally directed to compounds that can function as melanocortin (MC) receptor ligands. In this context, “ligands” are molecules that bind or form a complex with one or more of the MC receptors. This invention is also directed to compositions containing one or more compounds in combination with one or more pharmaceutically acceptable carriers, as well as to methods for treating conditions or disorders associated with MC receptors.

In one embodiment, this invention is directed to one or more compounds that have the following structure (I): 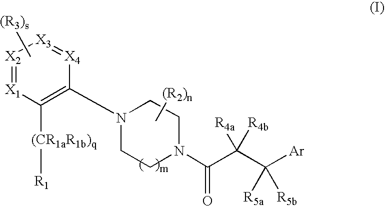
including stereoisomers, prodrugs, and pharmaceutically acceptable salts thereof, wherein m, n, q, s, R₁, R₁ₐ, R_{1b}, R₂, R₃, R₄ₐ, R_{4b}, R₅ₐ, R_{5b}, X₁, X₂, X₃, X₄ and Ar are as defined herein.

The compounds of this invention may have utility over a broad range of therapeutic applications, and may be used to treat disorders or illnesses, including (but not limited to) eating disorders, obesity, inflammation, pain, chronic pain, skin disorders, skin and hair coloration, sexual dysfunction, dry eye, acne, anxiety, depression, and/or Cushing's disease. A representative method of treating such a disorder or illness includes administering a pharmaceutically effective amount of a compound of this invention, typically in the form of a pharmaceutical composition, to an animal (also referred to herein as a “patient”, including a human) in need thereof. The compound may be an antagonist or agonist or may stimulate a specific melanocortin receptor while functionally blocking a different melanocortin receptor. Accordingly, in another embodiment, pharmaceutical compositions are disclosed containing one or more compounds of this invention in combination with a pharmaceutically acceptable carrier.

In one embodiment, the compounds are agonists to one or more MC receptors, and are useful in medical conditions where a melanocortin receptor agonist is beneficial. For example, the compounds may be utilized as MC4 receptor agonists or MC3 receptor agonists. Alternatively, the compounds may have mixed activity on the MC3 receptor and MC4 receptor, and may even function as an agonist to one receptor and and an antagonist to the other. In this context, the compounds may be used to treat obesity, erectile and/or sexual dysfunction, or diabetes mellitus.

In another embodiment, the compounds may serve as antagonists to either the MC3 receptor or MC4 receptor. Such antagonists may have beneficial therapeutic effects, especially in the treatment of cachexia or wasting disease associated with cancer, AIDS, failure to thrive syndrome, and diseases associated with aging and senility. In other embodiments, the compounds are MC4 receptor specific antagonists for treatment of cachexia or wasting disease associated with cancer, AIDS, failure to thrive syndrome, and diseases associated with aging and senility.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, in one embodiment the present invention is generally directed to compounds having the following structure (I): 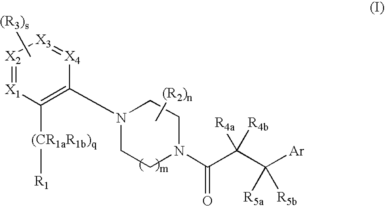
or a pharmaceutically acceptable salt, ester, solvate, stereoisomer, or prodrug thereof,
wherein:
   Ar is aryl, substituted aryl, heteroaryl, or substituted heteroaryl;
   X₁, X₂, X₃ and X₄ are independently CH or N, with the proviso that no more than two of X₁, X₂, X₃ and X₄ are N, and with the further proviso that when two of X₁, X₂, X₃ and X₄ are N either X₁ and X₃ are both N or X₂ and X₄ are both N;
   R₁ is Y₁—Y₂)—NR₆R₇, —NR₈C(═O)R₉, —NR₈S(O)ₚR₁₀, —NR₈C(═O)OR₁₁, imidazolyl, triazolyl, oxazolyl, or thiazolyl;
   Y₁ is —O—, —S— —NR₈—, —C(═O)—, —C(═O)O—, —OC(═O)—, —NR₈C(═O)O—, —NR₈C(═O)—, —C(═O)NR₈—, —NR₈S(═O)ₚ—, —S(═O)ₚ—, —S(═O)ₚNR₈—, or —NR₈C(═O)NR₈—;
   Y₂ is —(CR_{1c}R_{1d})ᵣ—;
   R₁ₐ, R_{1b}, R_{1c}, and R_{1d} are at each occurrence the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
   or R₁ₐ and R_{1b} taken together with the carbon atom to which they are attached form a homocycle or substituted homocycle;
   or R_{1c} and R_{1d} taken together with the carbon atom to which they are attached form a homocycle or substituted homocycle;
   or R_{1c} and R₆ taken together form a heterocycle or substituted heterocycle;
   R₂ is at each occurrence the same or different and independently alkyl or substituted alkyl, and where n is 0, 1, 2, 3 or 4 and represents the number of R₂ substituents;
   R₃ is at each occurrence the same or different and independently hydroxy, halogen, cyano, nitro, alkyl, haloalkyl, substituted alkyl, aryl, substituted aryl, heterocycle, or substituted heterocycle, and where s is 0, 1 or 2 and represents the number of R₃ substituents;
   R₄ₐ and R₅ₐ are the same or different and independently hydrogen, methyl, hydroxy, halogen, dialkylamino, substituted dialkylamino, heterocycle, or substituted heterocycle;
   R_{4b} band R_{5b} are the same or different and independently hydrogen or methyl;
   R₆ and R₇ are the same or different and independently are hydrogen, alkyl, substituted alkyl, aryl, substituted aryl, arylalkyl, substituted arylalkyl, heterocycle, substituted heterocycle, heterocyclealkyl, or substituted heterocyclealkyl,
   or R₆ and R₇ taken together with the nitrogen atom to which they are attached form a heterocyclic ring or a substituted heterocyclic ring;
   R₈ is at each occurrence the same or different and independently hydrogen, alkyl, substituted alkyl, —C(═O)R₉, or —SO₂R₁₀;
   R₉, R₁₀ and R₁₁ are are each occurrence the same or different and independently hydrogen, alkyl, substituted alkyl, aryl, or substituted aryl;
   m and p are the same or different and independently 0, 1 or 2; and
   q and r are the same or different and independently 0, 1, 2, 3 or 4.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂cyclohexyl, and the like; while unsaturated cyclic alkyls include cyclopentenyl, cyclohexenyl, —CH₂cyclohexenyl, and the like. Cyclic alkyls are also referred to herein as a “homocycle”, and include bicyclic rings in which a homocycle is fused to a benzene ring. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂phenyl), —(CH₂)₂phenyl, —(CH₂)₃phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

The term “substituted” as used herein means any of the above groups (i.e., alkyl, aryl, arylalkyl, heteroaryl, heteroarylalkyl, heterocycle and heterocyclealkyl) wherein at least one hydrogen atom is replaced with a substituent. In the case of an oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b}, —NRₐSO₂R_{b}, —C(═O)Rₐ, C(═O)ORₐ, C(═O)NRₐR_{b}, —OC(═O)NRₐR_{b}, ORₐ, —SRₐ, —SORₐ, —S(═O)₂Rₐ, —OS(═O)₂Rₐ, —S(═O)₂ORₐ, —CH₂S(═O)₂Rₐ, —CH₂S(═O)₂NRₐR_{b}, ═NS(═O)₂Rₐ, and —S(═O)₂NRₐR_{b}, wherein Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, carbocycle, substituted carbocycle, carbocyclealkyl or substituted carbocyclealkyl.

“Halogen” means fluoro, chloro, bromo or iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Alkylamino” and “dialkylamino” mean one or two alkyl moiety attached through a nitrogen bridge (i.e., —N-alkyl) such as methylamino, ethylamino, dimethylamino, diethylamino, and the like.

In more specific embodiments of structure (I), compounds of this invention have structure (II) when X₁, X₂, X₃ and X₄ are all CR₃ or CH, have either structure (III), (IV), (V) or (VI) when one X₁, X₂, X₃ and X₄ is N, and have structure (VII) or (VIII) when two of X₁, X₂, X₃ and X₄ are N:

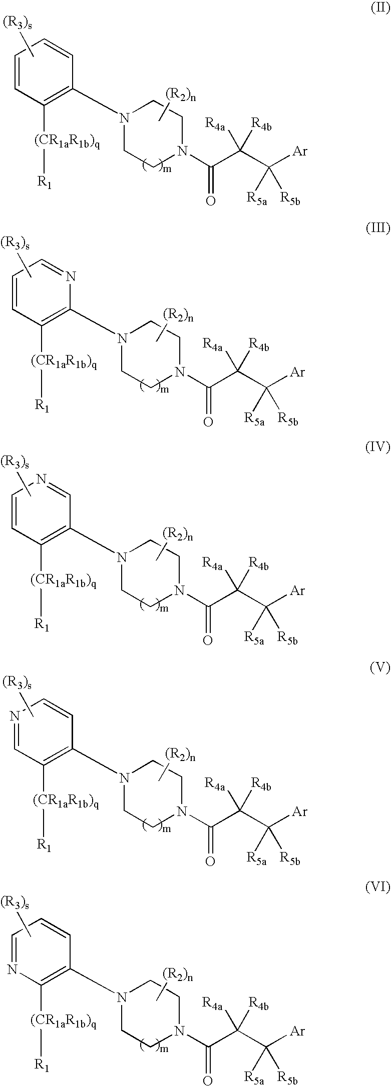

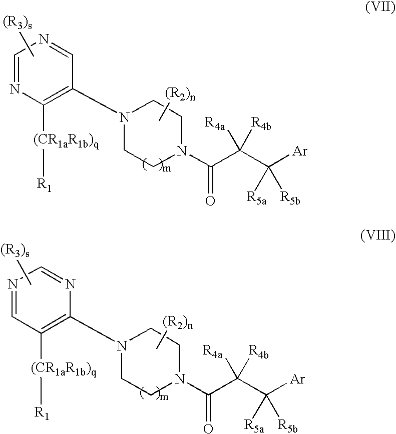

In more specific embodiments of structure (I), compounds of this invention have structure (IX) when R₁ is —(Y₁—Y₂)—NR₆R₇, have structure (X) when R₁ is —NR₈C(═O)R₉, have struture (XI) when R₁ is —NR₈S(O)ₚR₁₀, have structure (XII) when R₁ is —NR₈C(═O)OR₁₁, and have structures (XIII), (XIV) (XV) and (XVI) when R₁ is imidazolyl, triazolyl, oxazolyl and thiazolyl, respectively.

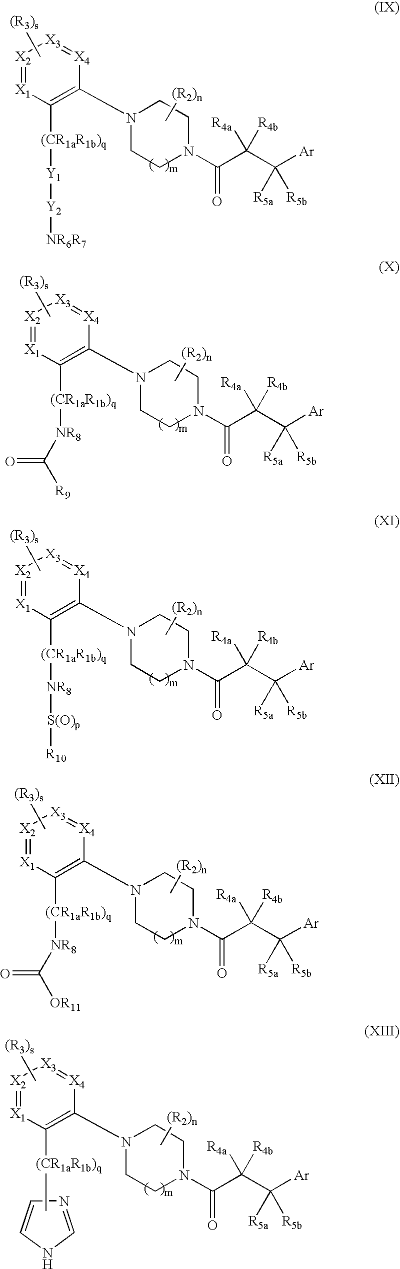

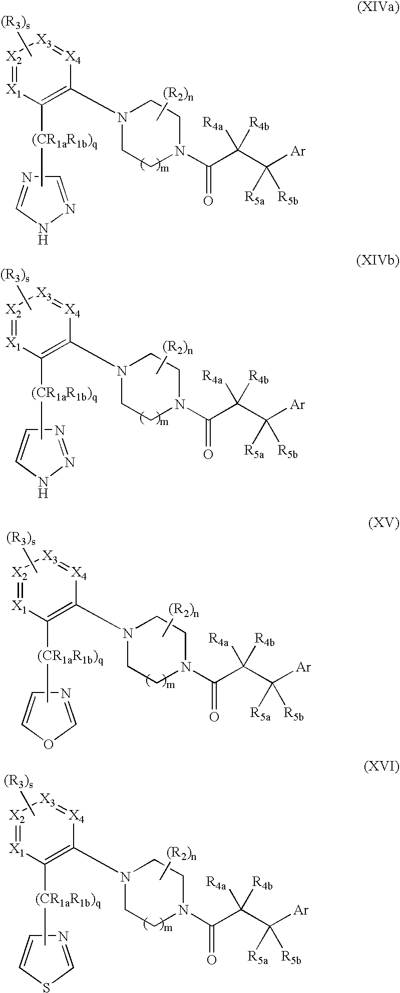

In a more specific embodiments of structure (IX), compounds of this invention have structure (IXa) when Y, is —NR₈C(O)— and r is 1, and have structure (IXb) when Y, is —C(═O)— and r is 0:

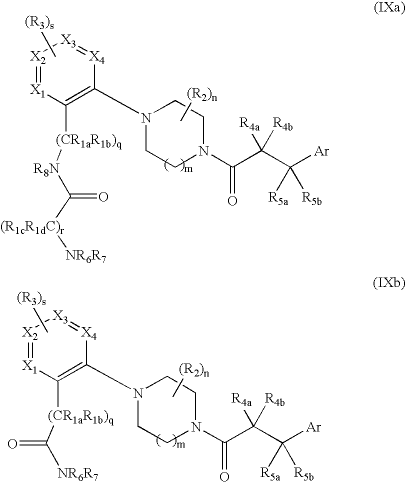

Further, in more specific embodiments of structure (XIII), compounds of this invention have structure (XIII) when R₁ is 1-imidazolyl, and have structure (XIII) when R₁ is 2-imidazolyl.

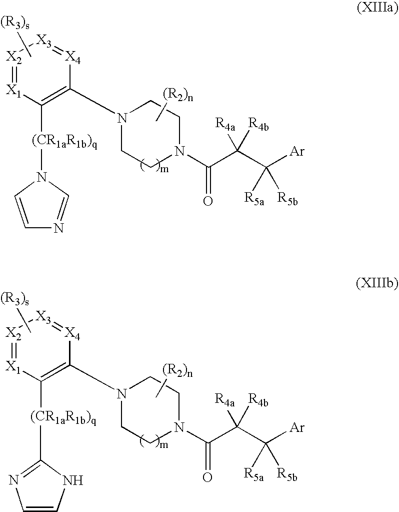

Further representative embodiments of R₁ include (but are not limited to) the following: —C(═O)NR₆R₇, —OC(═O)NR₆R₇, —NR₈C(═O)R₉, —NR₈S(═O)ₚR₁₀, —NR₈C(═O)OR₁₁, —S(═O)ₚNR₆R₇, —NR₈C(═O)NR₆R₇, —NR₈S(═O)ₚNR₆R₇, —O—(CR_{1c}R_{1d})ᵣNR₆R₇, —S—(CR_{1c}R_{1d})ᵣNR₆R₇, —C(═O)—(CR_{1c}R_{1d})ᵣNR₆R₇, —S(═O)ₚ—(CR_{1c}R_{1d})ᵣNR₆R₇, —C(═O)O—(CR_{1c}R_{1d})ᵣNR₆R₇, —NR₈—C(═O)—(CR_{1c}R_{1d})ᵣNR₆R₇, —C(═O)—NR₈—(CR_{1c}R_{1d})ᵣNR₆R₇, —OC(═O)O—(CR_{1c}R_{1d})ᵣNR₆R₇, —NR₈—C(═O)O—(CR_{1c}R_{1d})ᵣNR₆R₇, —NR₈C(═O)—NR₈—(CR_{1c}R_{1d})ᵣNR₆R₇, and —NR₈—(CR_{1c}R_{1d})ᵣNR₆R₇.

The compounds of the present invention may be prepared by known organic synthesis techniques, including the methods described in more detail in the following Reaction Scheme and Examples. Piperazine subunits of this invention are commercially available (including those having a bridging heterocycle or subsituted heterocycle groups), are known in the literature, and/or may be synthesized from extensions of known methods. Furthermore, compounds of the present invention may be synthesized by a number of methods, both convergent and sequential, utilizing solution or solid phase chemistry.

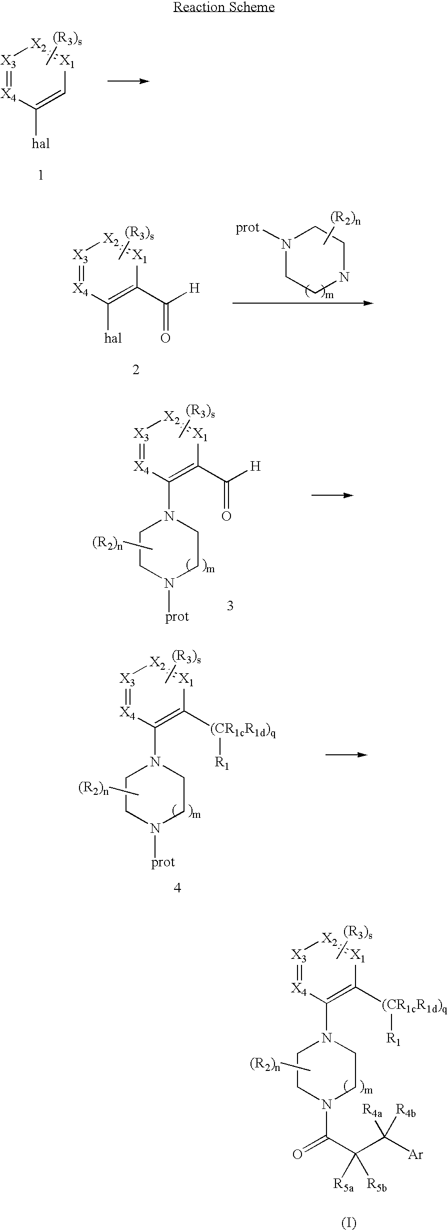

Aryl halide 1 may undergo a deprotonation with a reagent such as lithium diisopropylamide following by quenching with DMF to give 2. Alternatively, 2 may be obtained commercially. A replacement reaction of 2 with a heterocycle such as piperazine or substituted piperazine gives 3. The aldehyde functionality of 3 may be converted to compound 4 through techniques as described herein or known to those skilled in the art. The piperazine of compound 4 may be optionally substituted to give structure (I).

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Compounds of structure (I) may also possess axial chirality which may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be evaluated for their ability to bind to a MC receptor by techniques known in this field. For example, a compound may be evaluated for MC receptor binding by monitoring the displacement of an iodonated peptide ligand, typically [¹²⁵I]-NDP-α-MSH, from cells expressing individual melanocortin receptor subtypes. To this end, cells expressing the desired melanocortin receptor are seeded in 96-well microtiter Primaria-coated plates at a density of 50,000 cells per well and allowed to adhere overnight with incubation at 37° C. in 5% CO₂. Stock solutions of test compounds are diluted serially in binding buffer (D-MEM, 1 mg/ml BSA) containing [¹²⁵I]-NDP-α-MSH (10⁵ cpm/ml). Cold NDP-α-MSH is included as a control. Cells are incubated with 50 μl of each test compound concentration for 1 hour at room temperature. Cells are gently washed twice with 250 μl of cold binding buffer and then lysed by addition of 50 μl of 0.5 M NaOH for 20 minutes at room temperature. Protein concentration is determined by Bradford assay and lysates are counted by liquid scintillation spectrometry. Each concentration of test compound is assessed in triplicate. IC₅₀ values are determined by data analysis using appropriate software, such as GraphPad Prizm, and data are plotted as counts of radiolabeled NDP-MSH bound (normalized to protein concentration) versus the log concentration of test compound.

In addition, functional assays of receptor activation have been defined for the MC receptors based on their coupling to Gₛ proteins. In response to POMC peptides, the MC receptors couple to G_{S} and activate adenylyl cyclase resulting in an increase in cAMP production. Melanocortin receptor activity can be measured in HEK293 cells expressing individual melanocortin receptors by direct measurement of cAMP levels or by a reporter gene whose activation is dependent on intracellular cAMP levels. For example, HEK293 cells expressing the desired MC receptor are seeded into 96-well microtiter Primaria-coated plates at a density of 50,000 cells per well and allowed to adhere overnight with incubation at 37° C. in 5% CO_{2.} Test compounds are diluted in assay buffer composed of D-MEM medium and 0.1 mM isobutylmethylxanthine and assessed for agonist and/or antagonist activity over a range of concentrations along with a control agonist x-MSH. At the time of assay, medium is removed from each well and replaced with test compounds or α-MSH for 30 minutes at 37° C. Cells are harvested by addition of an equal volume of 100% cold ethanol and scraped from the well surface. Cell lysates are centrifuged at 8000×g and the supernatant is recovered and dried under vacuum. The supernatants are evaluated for cAMP using an enzyme-linked immunoassay such as Biotrak, Amersham. EC₅₀ values are determined by data analysis using appropriate software such as GraphPad Prizm, and data are plotted as cAMP produced versus log concentration of compound.

As mentioned above, the compounds of this invention function as ligands to one or more MC receptors, and are thereby useful in the treatment of a variety of conditions or diseases associated therewith. In this manner, the ligands function by altering or regulating the activity of an MC receptor, thereby providing a treatment for a condition or disease associated with that receptor. In this regard, the compounds of this invention have utility over a broad range of therapeutic applications, and may be used to treat disorders or illnesses, including (but not limited to) eating disorders, cachexia, obesity, diabetes, metabolic disorders, inflammation, pain, skin disorders, skin and hair coloration, male and female sexual dysfunction, erectile dysfunction, dry eye, acne and/or Cushing's disease.

The compounds of the present invention may also be used in combination therapy with agents that modify sexual arousal, penile erections, or libido such as sildenafil, yohimbine, apomorphine or other agents. Combination therapy with agents that modify food intake, appetite or metabolism are also included within the scope of this invention. Such agents include, but are not limited to, other MC receptor ligands, ligands of the leptin, NPY, melanin concentrating hormone, serotonin or B₃ adrenergic receptors.

In another embodiment, pharmaceutical compositions containing one or more compounds of this invention and a pharmaceutically acceptable carrier or diluent are disclosed. For the purposes of administration, the compounds of the present invention may be formulated as pharmaceutical compositions. Pharmaceutical compositions of the present invention comprise a pharmaceutically effective amount of a compound of structure (I) and a pharmaceutically acceptable carrier and/or diluent. Thus, the compound is present in the composition in an amount which is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating a condition associated with the activity of an MC receptor. Such methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration, not limitation.

### EXAMPLES

Aqueous Work Up

The reaction mixture was concentrated under a stream of nitrogen, taken up in dichloromethane, washed with aqueous sodium bicarbonate, and again concentrated. Final compounds were dissolved in methanol and filtered prior to preparative HPLC purification.

HPLC Columns and Gradients

Analytical HPLC columns were BHK laboratories ODS/0/13 30×75 mm, 5 μm, 120 A; the standard gradient was 1 mL/min 10-90% CH₃CN in water over 2 minutes, then 90% CH3CN for 1 minute. Constant percentage of 0.1% TFA was added.

Prep HPLC Column

YMC AQ, 5 μm, 120 A20, 20×50 mm cartridges

Analytical Procedures

A—Analytical HPLC-MS (LC-MS)

HP 1100 series: equipped with an auto-sampler, an UV detector (220 nM and 254 nM), a MS detector (electrospray);
HPLC column: YMC ODS AQ, S-5, 5μ, 2.0×50 mm cartridge;
HPLC gradients: 1.5 mL/minute, from 10% acetonitrile in water to 90% acetonitrile in water in 2.5 minutes, maintaining 90% for 1 minute.
   B—Prep. HPLC-MS
Gilson HPLC-MS equipped with Gilson 215 auto-sampler/fraction collector, an UV detector and a ThermoFinnigan AQA Single QUAD Mass detector (electrospray);
HPLC column: BHK ODS-O/B, 5μ, 30×75 mm
HPLC gradients: 35 mL/minute, 10% acetonitrile in water to 100% acetonitrile in 7 minutes, maintaining 100% acetonitrile for 3 minutes.
   C—Analytical HPLC-MS (LC-MS)
HP 1100 series: equipped with an auto-sampler, an UV detector (220 nM and 254 nM), a MS detector (electrospray);
HPLC column: YMC ODS AQ, S-5, 5μ, 2.0×50 mm cartridge;
HPLC gradient: 1.5 mL/minute, from 10% acetonitrile in water to 90% acetonitrile in water in 2.5 minutes, maintaining 90% for 1 minute. Both acetonitrile and water have 0.025% TFA.
   D—Analytical HPLC-MS (LC-MS)
HP 1100 series: equipped with an auto-sampler, an UV detector (220 nM and 254 nM), a MS detector (electrospray);
HPLC column: Phenomenex Synergi-Max RP, 2.0×50 mm column;
HPLC gradient: 1.0 mL/minute, from 5% acetonitrile in water to 95% acetonitrile in water in 13.5 minutes, maintaining 95% for 2 minute. Both acetonitrile and water have 0.025% TFA.
   E—Analytical HPLC-MS (LC-MS)
HP 1100 series: equipped with an auto-sampler, an UV detector (220 nM and 254 nM), a MS detector (electrospray);
HPLC column: XTerra MS, C₁₈, 5μ, 3.0×250 mm cartridge;
HPLC gradient: 1.0 mL/minute, from 5% acetonitrile in water to 90% acetonitrile in water in 47.50 minutes, maintaining 99% for 8.04 minutes. Both acetonitrile and water have 0.025% TFA.
   F—Analytical HPLC-MS (LC/MS)
Gilson 333/334 series: equipped with a Gilson 215 Liquid-Handler, a Gilson UV/VIS-156 UV detector (220 nM and 254 nM) and Finnigan AQA Mass Spec (ElectroSpray);
HPLC column: BHK Alpha, C-18, 5μ, 120A, 4.6×150 mm cartridge (PN: OB511546);
HPLC gradient: 3.6 mL/minute, maintaining 10% acetonitrile in water for 1 minute. Increasing from 10% acetonitrile in water to 90% acetonitrile in water over 12 minutes. Then increasing to 99% in 0.1 minutes and maintaining for 1.5 minutes. Both acetonitrile and water have 0.05% TFA.
   G—Analytical HPLC-MS (SFC-MS)
HP 1100 series: equipped with an auto-sampler, an UV detector (220 nM and 254 nM), a MS detector (electrospray) and FCM 1200 CO₂ pump module;
HPLC column: Berger Pyridine, PYR 60A, 6μ, 4.6×150 mm column;
HPLC gradient: 4.0 mL/minute, 120 bar; from 10% methanol in supercritical CO₂ to 60% methanol in supercritical CO₂ in 1.67 minutes, maintaining 60% for 1 minute. Methanol has 1.5% water. Backpressure regulated at 140 bar.
   H—Analytical HPLC (HPLC)
Shimadzu SIL-10A series: equipped with an auto-sampler and UV detector (220 nM and 254 nM);
HPLC column: ZORBAX SB-C18, 5μ, 4.6×250 mm cartridge (PN: 880975-902);
HPLC gradient: 2.0 mL/minute, maintaining 5% acetonitrile in water for 4 minutes then to 10% acetonitrile in 0.1 min and 10% acetonitrile in water to 95% acetonitrile in water in 46 minutes, then increasing to 99% in 0.1 minutes and maintaining for 10.8 minutes. Both acetonitrile and water have 0.025% TFA.
   1—Analytical HPLC (HPLC)
HP 1100 series: equipped with an auto-sampler and UV detector (220 nM and 254 nM);
HPLC column: Waters Symetry, C-8, 5μ, 4.6×150 mm cartridge (PN: WAT045995);
HPLC gradient: 2.8 mL/minute, maintaining 5% acetonitrile in water for 1 minute. Increasing to 10% acetonitrile in water in 0.1 minutes. Then increasing to 90% acetonitrile in water in 15 minutes. Then increasing to 99% in 0.1 minutes and maintaining for 2.4 minutes. Both acetonitrile and water have 0.05% TFA.

### Example 1

### 2-{4-[3-(2,4-DICHLOROPHENYL)-2-METHYLPROPIONYL]-1-PIPERAZINYL}-3-{1S-[2-(METHYLAMINO)ACETAMIDO]-3-METHYLBUTYL}PYRIDINE

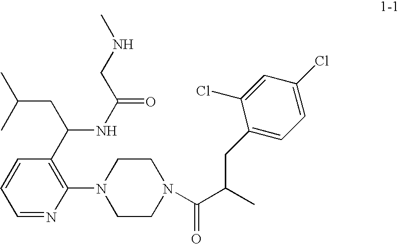

### Step 1A. 2-Bromo-3-formylpyridine 1a

Lithium diisopropylamide (131 mL, 262 mmol, 2M in THF) was added to a stirring solution of 2-bromopyridine (25 mL, 262 mmol) in THF (208 mL) at −78° C. under nitrogen. The reaction mixture was allowed to stir at −78° C. for 2 hours and then a solution of DMF (20.3 mL, 262 mmol) in THF (104 mL) was added. After the addition, the reaction mixture was allowed to warm to r.t. and was neutralized by adding a saturated solution of ammonium chloride. The crude product was extracted with ethyl acetate (3×200 mL), the organic layers were combined, dried over anhydrous Na₂SO₄, filtered, and solvent removed in vacuo. The residue was purified by column chromatography on silica using 15% ethyl acetate/hexanes as the eluent (R_{f}=0.3). Compound 1a was recovered in 19% yield as a yellow oil (9.4 g, 50.5 mmol).

### Step 1B. 2-(4-Boc-piperazinyl)-3-formylpyridine 1b

To the reaction flask, 2-bromo-3-formylpyridine 1a (9.4 g, 50.5 mmol) was dissolved in DMF (100 mL) along with diisopropylethylamine (8.8 mL, 50.5 mmol) and 1-Boc-piperazine (9.4 g, 50.5 mmol). The reaction mixture was heated at 100° C. for 8 hours then cooled to room temperature and quenched with saturated NaHCO₃ (150 mL). The crude product was extracted with ethyl acetate (3×100 mL), the organic layers were combined, dried over anhydrous Na₂SO₄, filtered, and solvent removed in vacuo. The residue was purified by column chromatography on silica using 25% ethyl acetate/hexanes as the eluent (R_{f}=0.3). Compound 1b was recovered in 67% yield as a yellow solid (9.8 g, 33.5 mmol).

### Step 1C. 2-(4-Boc-piperazinyl)-3-(S-tert-butalsulfinyliminomethylidene)pyridine 1c

2-(4-Boc-piperazinyl)-3-formylpyridine 1b (3 g, 10.3 mmol) was dissolved in THF (51 mL) along with S-2-methyl-2-propanesulfinamide (1.4 g, 11.3 mmol) and titanium (IV) ethoxide (8.6 mL, 41.2 mmol). The reaction mixture was allowed to stir at room temperature for 8 hours then saturated NaCl solution (20 mL) was added. The reaction mixture was filtered and the solid was washed with ethyl acetate (3×75 mL). The organic layer was collected, dried over anhydrous Na₂SO₄, filtered, and solvent removed in vacuo. Compound 1c was isolated as a yellow solid in quantitative yield without further purification (4.1 g, 10.3 mmol).

### Step 1 D. 2-(4-Boc-piperazinyl)-3-[1S-(S-tert-butylsulfinamino)-3-methylbutyl]pyridine 1d

2-(4-Boc-piperazinyl)-3-(S-tert-butylsulfinyliminomethylidene)pyridine 1c (4.1 g, 10.3 mmol) in THF (30 mL) was cooled to −40° C. and Me₃Al (15.45 mL, 30.9 mmol) was added. The reaction mixture was allowed to stir at −40° C. under nitrogen atmosphere for 20 minutes then cooled to −78° C. To the reaction mixture, isobutyl lithium (12.9 mL, 20.6 mmol, 1.6 M in heptane) was added slowly at −78° C. After the addition was complete, the reaction mixture was warmed to room temperature and carefully quenched with water. The mixture was concentrated under vacuum and diluted with dichloromethane (150 mL). The organic layer was then washed with saturated NaHCO₃ solution (2×100 mL), saturated NaCl solution (100 mL), dried over anhydrous MgSO₄, filtered, and solvent removed in vacuo. The residue was purified by column chromatography on silica using 75% ethyl acetate/hexanes as the eluent (R_{f}=0.3). Compound 1d was recovered in 60% yield as a yellow solid (2.8 g, 6.15 mmol).

### Step 1E. 2-{4-[3-(2,4-dichlorophenyl)-2-methylpropionyl]piperazinyl}-3-(1S-amino-3-methylbutyl)pyridine 1e

2-(4-Boc-piperazinyl)-3-[1S-(S-tert-butylsulfinamino)-3-methylbutyl]pyridine 1d (452.6 mg, 1 mmol) was allowed to stir at room temperature for 1.5 hours in 20% TFA/DCM mixture. The reaction was quenched with saturated NaHCO₃ solution (5 mL). The organic layer was washed additionally washed with saturated NaHCO₃ solution (2×10 mL), saturated NaCl solution (10 mL), dried over anhydrous MgSO₄, filtered, and solvent removed in vacuo. The deprotected piperazine intermediate was recovered in quantitative yield. A small portion of this piperazine intermediate (35.2 mg, 0.1 mmol) was dissolved in dichloromethane (0.5 mL) along with HOBt (13.5 mg, 0.1 mmol) and 3-(2,4-dichlorophenyl)-2-methylpropionic acid (25 mg, 0.1 mmol). The reaction mixture was allowed to stir at room temperature for 10 minutes then 1-(3-dimethylaminopropyl) 3-ethylcarbodiimide hydrochloride (EDC, 19.2 mg, 0.1 mmol) was added. The reaction was then stirred for an additional 8 hours at room temperature followed by quenching with saturated NaHCO₃ solution. The organic layer was separated, washed with saturated NaCl solution (2 mL), dried over anhydrous MgSO₄, filtered, and solvent removed in vacuo. The resulting residue was dissolved in MeOH (2 mL) and 0.2M HCl/ether (1 mL) was added. The reaction was stirred at room temperature for 1 hour then solvent was removed under a stream of nitrogen. The residue was purified by prep HPLC to yield 1e in 26% yield as the TFA salt (15 mg, 0.026 mmol).

### Step 1F. 2-{4-[3-(2,4-Dichlorophenyl)-2-methylpropionyl]piperazinyl}-3-{1S-[2-(methylamino)acetamido]-3-methylbutyl}pyridine 1-1

2-{4-[3-(2,4-dichlorophenyl)-2-methylpropionyl]piperazinyl}-3-(1S-amino-3-methylbutyl)pyridine trifluoroacetic acid salt 1e (580 mg, 1 mmol) was dissolved in dichloromethane and saturated NaHCO₃ solution (5 mL). The organic layer was additionally washed with saturated NaHCO₃ solution (2×10 mL), saturated NaCl solution (10 mL), dried over anhydrous MgSO₄, filtered, and solvent removed in vacuo. The intermediate (47 mg) was dissolved in dichloromethane (0.5 mL) along with HOBt (13.5 mg, 0.1 mmol) and 3-(2,4-dichlorophenyl)-2-methylpropionic acid (25 mg, 0.1 mmol). The reaction mixture was allowed to stir at room temperature for 10 minutes then EDC (19.2 mg, 0.1 mmol) was added. The reaction was then stirred for an additional 8 hours at room temperature followed by quenching with saturated NaHCO₃ solution. The organic layer was separated, washed with saturated NaCl solution (2 mL), dried over anhydrous MgSO₄, filtered, and solvent removed in vacuo. The resulting residue was allowed to stir at room temperature for 1.5 hours in 20% TFA/DCM mixture, then solvent was removed under a stream of nitrogen. The crude product was purified by prep HPLC to yield 1-1 as the TFA salt.

By the above procedures, the compounds of the following Tables 1A and 1B were prepared.

TABLE 1A

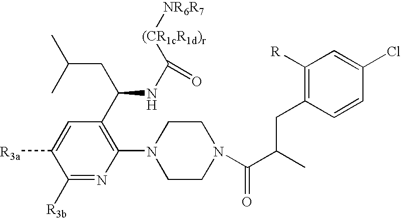

| Cpd | —(CR_{1c}R_{1d})r—NR₆R₇ | R | R₃ₐ | R_{3b} | [MH]⁺ | MW | T_{R} |
|---|---|---|---|---|---|---|---|
| 1-1 | MeNHCH₂— | Cl | H | H | 533.8 | 534.53 | 8.345 (1) |
| 1-2 | NH₂CH₂— | Cl | H | H | 519.7 | 520.50 | 8.115 (1) |
| 1-3 | NH₂CH₂— | MeO— | H | H | 516.2 | 516.08 | 7.270 (1) |
| 1-4 | MeNHCH₂— | MeO— | H | H | 530.2 | 530.11 | 7.420 (1) |
| 1-5 | NH₂CH₂CH₂— | MeO— | H | H | 530.2 | 530.11 | 7.250 |
| 1-6 | R—NH₂CH(CH₃)— | MeO— | H | H | 530.2 | 530.11 | 7.390 |
| 1-7 | NH₂CH₂— | H | H | H | 486.2 | 486.06 | 7.020 |
| 1-8 | MeNHCH₂— | H | H | H | 500.2 | 500.08 | 7.140 |
| 1-9 | NH₂CH₂CH₂— | H | H | H | 500.2 | 500.08 | 6.920 |
| 1-10 | NH₂CH(CH₃)— | H | H | H | 500.2 | 500.08 | 6.970 |
| 1-11 | NH₂CH₂— | Cl | F | H | 537.9 | 538.49 | 6.841 |
| 1-12 | MeNHCH₂— | Cl | F | H | 552.0 | 552.52 | 7.107 |
| 1-13 | Me₂NCH₂— | Cl | F | H | 566.0 | 552.52 | 7.184 |
| 1-14 | NH₂CH₂CH₂— | Cl | F | H | 552.0 | 552.52 | 7.002 |
| 1-15 | NH₂CH(CH₃)— | Cl | F | H | 552.0 | 552.52 | 6.926 |
| 1-16 | MeNHCH₂CH₂— | Cl | F | H | 566.2 | 566.54 | 6.612 |
| 1-17 | NH₂CH₂— | Cl | H | Me | 534.4 | 534.53 | 4.887 |
| 1-18 | MeNHCH₂— | Cl | H | Me | 548.4 | 548.55 | 5.130 |
| 1-19 | NH₂CH₂CH₂— | Cl | H | Me | 548.4 | 548.55 | 4.951 |
| 1-20 | Me₂NCH₂— | Cl | H | Me | 562.5 | 562.58 | 5.096 |
| 1-21 | NH₂CH₂— | H | H | Me | 500.4 | 500.08 | 4.812 |
| 1-22 | NH₂CH₂— | MeO— | H | Me | 530.5 | 530.11 | 4.907 |
| 1-23 | MeNHCH₂— | MeO— | H | Me | 544.4 | 544.14 | 4.822 |
| 1-24 | NH₂CH₂CH₂— | MeO— | H | Me | 544.4 | 544.14 | 4.802 |
| 1-25 | Me₂NCH₂— | MeO— | H | Me | 558.4 | 558.16 | 5.026 |
| 1-26 | Me₂NCH₂CH₂— | Cl | F | H | 580.0 | 580.57 | 5.809 |
| 1-27 | NH₂CH₂— | H | F | H | 504.0 | 504.05 | 5.345 |
| 1-28 | MeNHCH₂— | H | F | H | 518.0 | 518.07 | 5.394 |
| 1-29 | NH₂CH₂— | MeO— | F | H | 534.0 | 534.07 | 5.467 |
| 1-30 | MeNHCH₂— | MeO— | F | H | 548.0 | 548.10 | 5.404 |
| 1-31 | NH₂CH₂CH₂— | MeO— | F | H | 548.0 | 548.10 | 5.490 |
| 1-32 | Me₂NCH₂— | MeO— | F | H | 562.0 | 562.13 | 5.453 |
| 1-33 | Me₂NCH₂CH₂— | MeO— | F | H | 576.1 | 576.15 | 5.437 |
| 1-34 | Me₂NCH₂CH₂— | Cl | H | Me | 576.1 | 576.61 | 5.758 |
| 1-35 | MeNHCH₂CH₂— | Cl | H | Me | 562.1 | 562.58 | 5.819 |
| 1-36 | Me₂NCH₂— | Cl | H | H | 548.0 | 548.55 | 5.662 |
| 1-37 | NH₂CH₂CH₂— | Cl | H | H | 534.0 | 534.53 | 5.437 |
| 1-38 | MeNHCH₂CH₂— | Cl | H | H | 548.1 | 548.55 | 5.479 |
| 1-39 | Me₂NCH₂CH₂— | Cl | H | Me | 577.8 | 576.61 | 6.021 |
| 1-40 | Me₂NCH₂CH₂— | Cl | H | Me | 577.8 | 576.61 | 5.754 |

TABLE 1B

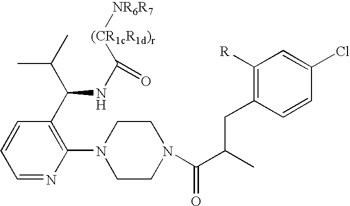

| Cpd | —(CR_{1c}R_{1d})r—NR₆R₇ | R | [M + H]⁺ | MW | t_{R} |
|---|---|---|---|---|---|
| 1-41 | NH₂CH₂— | MeO— | 502.0 | 502.06 | 5.392 |
| 1-42 | NH₂CH₂— | Cl | 506.0 | 506.48 | 5.104 |
| 1-43 | MeNHCH₂— | Cl | 520.0 | 520.50 | 5.265 |
| 1-44 | NH₂CH₂CH₂— | Cl | 520.0 | 520.50 | 5.235 |
| 1-45 | MeNHCH₂CH₂— | Cl | 534.0 | 534.53 | 5.228 |

### Example 2

### 1-{4-[3-(2,4-DICHLOROPHENYL)-2R-(2-oxo-1-PYRROLIDINYL)PROPIONYL]-1-PIPERAZINYL}-2-{1 S-[3-AMINOPROPIONYLAMIDO]-3-METHYLBUTYL}-6-FLUOROBENZENE

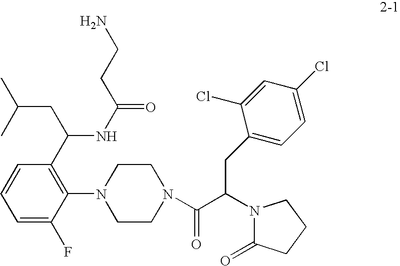

### Step 2A. 2-[4-(tert-Butoxycarbonyl)-1-piperazinyl]-3-fluorobenzaldehyde 2a

To a solution of 2,3-difluoro-benzaldehyde (10.0 g, 70.4 mmol) and 1-BOC-piperazine (19.7 g, 106 mmol) in 140 mL of DMF was added K₂CO₃ (97.1 g, 704 mmol). The reaction mixture was heated and stirred at 110° C. After stirring overnight, the reaction mixture was cooled to room temperature and diluted with 200 mL of EtOAc. The mixture was filtered, and the filter was washed well with EtOAc (3×100 mL). The filtrate was washed with 5% aqueous HCl (100 mL) and the aqueous layer was extracted with EtOAc (3×50 mL). The combined organic layers were washed with H₂O (2×100 mL), brine (50 mL), dried (MgSO₄), and concentrated in vacuo. The residue was triturated with 3% EtOAc/Hexanes to give 2a as a yellow solid (21 g).

### Step 2B. (S)-N-{2-[4-(tert-Butoxycarbonyl)-1-piperazinyl]-3-fluoro-benzylidene}-t-butanesulfinamide 2b

To a THF (12 mL) solution of aldehyde 2a (1.44 g, 4.66 mmol) at room temperature was added Ti(OEt)₄ (tech. Grade, Ti 20%, contains excess ethanol, 4.1 mL, 18.6 mmol), (S)-(−)-2-methyl-2-propanesulfinamide (0.64 g, 5.12 mmol) and the mixture was stirred overnight. The reaction mixture was poured to a saturated aqueous NaCl solution (15 mL) at room temperature with vigorous stirring and the resulting suspension was filtered through Celite and the filter cake was washed with EtOAc (300 mL). After phase separation, the aqueous layer was extracted with EtOAc (30 mL) and the combined organic layers were dried over Na₂SO₄ and evaporated to give a residue which was purified by triturating with 5˜10% EtOAc/Hexanes triturating to give 1.90 g of 2b as a light yellow powder (99%).

### Step 2C. 2-[4-(tert-Butoxycarbonyl)-1-piperazinyl]-3-fluoro-1-[1S-(S-t-butanesulfinamido)-3-methylbutyl]benzene 2c

To a THF (46 mL) solution of 2b (4.29 g, 10.4 mmol) was added trimethylaluminum (2.0 M in toluene or heptane or hexane, 10.4 mL, 20.8 mmol) at −40° C. and the mixture was stirred for 20 minutes. The mixture was cooled to −78° C. and i-BuLi (1.6 M in heptane from Fluka, 13.0 mL, 20.8 mmol) was added into this mixture and the i-BuLi addition rate was controlled by syringe pump at 1.6 mL/h (larger scale and higher rate). After i-BuLi addition, the reaction mixture was stirred for 30 minutes at −78° C., quenched with a 5% aqueous HCl (50 mL) at −78° C., warmed to 10° C. and extracted with EtOAc (3×50 mL). The combined organic layers were washed with brine (30 mL), dried over Na₂SO₄ and evaporated to provide an oil which was purified by 1035% EtOAc/Hexanes chromatography to give 4.49 g of 2c as a white foam (92%).

### Step 2D. 2-{4-[3-(2,4-Dichlorophenyl)-2R-tert-Boc-amino-1-piperazinyl]-3-fluoro-1-[1 S-(S-t-butanesulfinamido)-3-methylbutyl]benzene 2d

To a dichloromethane (18 mL) solution of BOC-piperazine 2c (1.02 g, 2.17 mmol) was added TFA (4.5 mL) at 23° C. and the mixture was stirred for 45 minutes. The reaction mixture was basified with saturated aqueous NaHCO₃ solution (100 mL) and extracted with EtOAc (2×100 mL). The organic layer was dried over Na₂SO₄ and evaporated to provide the piperazine as a white foam, which was dissolved in DMF/dichloromethane (1:3, 12 mL). To this solution was added NaHCO₃ (0.365 g, 4.34 mmol), BOC-D-dichloro-Phe (0.871 g, 2.61 mmol), HOBt (0.352 g, 2.61 mmol), EDCI (0.500 g, 2.61 mmol) sequentially. The reaction mixture was stirred overnight at room temperature. The mixture was diluted with EtOAc (60 mL), washed with 5% aqueous HCl (15 mL), saturated aqueous NaHCO₃ (15 mL), brine (15 mL), and dried (Na₂SO₄). The solution was concentrated in vacuo to provide a residue which was purified by flash column chromatography (30˜60% EtOAc in Hexanes) to provide compound 2d as a white solid (1.295 g, 87%).

### Step 2E. 2-{4-[3-(2,4-Dichlorophenyl)-2R-(2-oxo-1-pyrrolidinyl)-1-piperazinyl]-3-fluoro-1-[1S-(S-t-butanesulfinamido)-3-methylbutyl]benzene 2e

To a dichloromethane (4 mL) solution of 2-{4-[3-(2,4-dichlorophenyl)-2R-tert-Boc-amino-1-piperazinyl]-3-fluoro-1-[1S-(S-t-butanesulfinamido)-3-methylbutyl]benzene 2d (338 mg, 0.494 mmol) was added TFA (1 mL) at 23° C. and the mixture was stirred for 60 minutes. The reaction mixture was basified with saturated aqueous NaHCO₃ solution (30 mL) and extracted with EtOAc (2×30 mL). The organic layer was dried over Na₂SO₄ and evaporated to provide the free amine as white foam, which was dissolved in 1,2-dichloroethane (5 mL). Acetic acid (118 μL, 1.98 mmol) and succinic semialdehyde (374 μL 15 wt % solution in water, 0.592 mmol) were added and the mixture was stirred for 30 minutes. NaBH(OAc)₃ (220 mg, 0.987 mmol) was added and the reaction mix was stirred for 24 hours. HPLC showed the reductive amination was completed and the ratio of lactam product (M.W.=653) and carboxylic acid product (M.W.=671) was 1 to 1. The reaction was stopped with 10 mL of brine and extracted with 40 mL EtOAc. The extract was dried and the solvent was removed in vacuo to provide a residue which was dissolved in DMF/MC (1:3, 10 mL). To this solution was added NaHCO₃ (0.083 g, 0.988 mmol), HOBt (0.080 g, 0.592 mmol), and EDCI (0.113 g, 0.592 mmol), sequentially. The reaction mixture was stirred overnight at room temperature. The mixture was diluted with EtOAc (60 mL), washed with 5% aqueous HCl (10 mL), saturated aqueous NaHCO₃ (10 mL), brine (10 mL), and was dried (Na₂SO₄). The organic layer was concentrated in vacuo to provide crude product, which was purified by flash column chromatography (40˜70% EtOAc in Hexanes) to provide 2e as a white solid (0.239 g, 74%).

### Step 2F. 2-{4-[3-(2,4-Dichlorophenyl)-2R-(2-oxo-1-pyrrolidinyl)-1-piperazinyl]-3-fluoro-1-[1S-(3-aminopropionylamido)-3-methylbutyl]benzene 2-1

To a MeOH (4 mL) solution of 2-{4-[3-(2,4-dichlorophenyl)-2R-(2-oxo-1-pyrrolidinyl)-1-piperazinyl]-3-fluoro-1-[1S-(S-t-butanesulfinamido)-3-methylbutyl]benzene 2e (239 mg, 0.366 mmol) was added 2 equiv. HCl (183 μL, 4 N HCl in dioxane) at 23° C. and the mixture was stirred for 30 minutes. The excess HCl and MeOH were removed in vacuo to give free amine which was used for next reaction without purification. The free amine (34.0 mg, 0.0619 mmol) was dissolved in 1 mL of DMF/MC (1:3) followed by the addition of NaHCO₃ (13.0 mg, 0.155 mmol), HOBt (10.0 mg, 0.0742 mmol), and EDCI (11.9 mg, 0.0742 mmol), sequentially. The reaction mixture was stirred overnight at room temperature. The mixture was diluted with EtOAc (30 mL), washed with 5% aqueous HCl (10 mL), saturated aqueous NaHCO₃ (10 mL), brine (10 mL), and was dried (Na₂SO₄). The solution was concentrated in vacuo to provide a residue which was treated with 2 mL of TFA/MC (1:1) for 1 h. The excess of TFA and MC were removed in vacuo and the residue was purified by flash column chromatography (5˜10% MeOH/MC) to provide 2-1 as a white solid (35.7 mg, 93%).

By the above procedures, the compounds of the following Tables 2A and 2B were prepared.

TABLE 2A

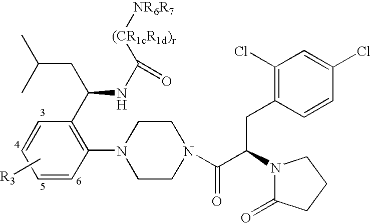

| Cpd. | R₃ | —(CR_{1c}R_{1d})ᵣ—NR₆R₇ | (MH³⁰ ) | M.W. | t_{R} |
|---|---|---|---|---|---|
| 2-1 | 6-F | —CH₂CH₂NH₂ | 620.1 | 620.59 | 9.610 (3) |
| 2-2 | 4-F | —CH₂NH₂ | 606.1 | 606.57 | 1.300 (4) |
| 2-3 | 4-F | —CH₂NHMe | 620.1 | 620.59 | 1.270 (4) |
| 2-4 | 4-F | —CH₂NMe₂ | 634.1 | 634.62 | 1.287 (4) |
| 2-5 | 4-F | —CH₂CH₂NH₂ | 620.1 | 620.59 | 1.316 (4) |
| 2-6 | 4-CF₃ | —CH₂CH₂NH₂ | 670.1 | 670.60 | 10.13 (3) |
| 2-7 | 4-F | —CH₂CH₂CH₂NH₂ | 634.1 | 634.62 | 1.230 (4) |
| 2-8 | 4-F | 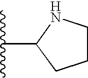 | 646.1 | 646.63 | 1.238 (4) |
| 2-9 | 4-F | 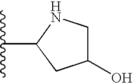 | 662.0 | 662.63 | 1.274 (4) |
| 2-10 | 4-F | (S)-CH(NH₂)Me | 620.0 | 620.59 | 1.277 (4) |
| 2-11 | 4-F | (R)-CH(NH₂)Me | 620.0 | 620.59 | 1.271 (4) |
| 2-12 | 4-F | —C(NH₂)Me₂ | 634.1 | 634.62 | 1.245 (4) |
| 2-13 | 4-Me | (R)-CH(NH₂)Me | 616.3 | 616.63 | 9.590 (3) |
| 2-14 | 4-Me | MeNHCH₂— | 616.3 | 616.63 | 9.420 (3) |
| 2-15 | 4-Me | Me₂NCH₂CH₂— | 644.4 | 644.68 | 9.730 (3) |
| 2-16 | H | Me₂NCH₂CH₂— | 630.1 | 630.66 | 5.345 (2) |
| 2-17 | H | H₂NCH₂CH₂— | 602.3 | 602.6 | 6.124 (2) |
| 2-18 | H | MeNHCH₂— | 602.3 | 602.60 | 6.104 (2) |
| 2-19 | 4-Me | MeNHCH₂CH₂— | 630.2 | 630.66 | 9.890 (3) |
| 2-20 | H | MeNHCH₂CH₂— | 616.3 | 616.63 | 5.186 (2) |

TABLE 2B

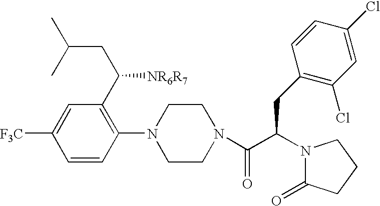

| Cpd. | R₃ | —NR₆R₇ | (MW⁺) | M.W. | t_{R} |
|---|---|---|---|---|---|
| 2-21 | 6-F | 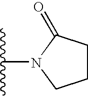 | 667.2 | 667.60 | 14.69 (3) |

### Example 3

### 1-{4-[3-(2,4-DICHLOROPHENYL)PROPIONYL]-1-PIPERAZINYL}-2-[1 S-(3-AMINOPROPIONYLAMIDO)-3-METHYLBUTYL]-4-TRIFLUOROMETHYLBENZENE

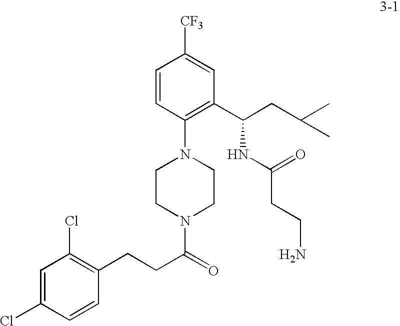

### Step 3A. 2-[4′-(tert-Butoxycarbonyl)-1-piperazinyl]-5-trifluoromethyl-benzaldehyde 3a:

To a solution of 2-fluoro-5-trifluoromethyl-benzaldehyde (110.0 mL, 68.7 mmol) and 1-BOC-piperazine (15.4 g, 82.4 mmol) in 140 mL of DMF was added K₂CO₃ (47.4 g, 344 mmol). The reaction mixture was heated and stirred at 120° C. for 10 hours. The reaction mixture was cooled to room temperature and diluted with 200 mL of EtOAc. The mixture was filtered, and the filter was washed well with EtOAc (3×50 mL). The filtrate was washed with 5% aqueous HCl (100 mL) and the aqueous layer was extracted with EtOAc (3×25 mL). The combined organic layers were washed with H₂O (2×40 mL), brine (50 mL), dried (MgSO₄), and concentrated in vacuo. The residue was triturated with hexanes (3×20 mL) to form a brown oil which slowly solidified to give 3a as a yellow solid (22.3 g, 92%).

### Step 3 B. 2-[4-(tert-Butoxycarbonyl)-1-piperazinyl]-5-trifluoromethyl-benzylidene}-t-butanesulfinamide 3b

To a THF (41 mL) solution of aldehyde 3a (3.29 g, 9.18 mmol) at room temperature was added Ti(OEt)₄ (tech. Grade, Ti ˜20%, contains excess ethanol, 9 mL, 36.7 mmol) and (S)-(−)-2-methyl-2-propanesulfinamide (1.26 g, 10.1 mmol) and the mixture was stirred overnight. The reaction mixture was poured into a saturated aqueous NaCl solution (30 mL) at room temperature with vigorous stirring and the resulting suspension was filtered through Celite and the filter cake was washed with EtOAc (500 mL). After phase separation, the aqueous layer was extracted with EtOAc (30 mL) and the combined organic layers were dried over Na₂SO₄ and evaporated to a residue which was purified by trituration with 5˜10% EtOAc/Hexanes to give 3b as a light yellow powder (4.2 g, 99%).

### Step 3C. 2-[4-(tert-Butoxycarbonyl)-1-piperazinyl]-1-[1S-(S-t-butanesulfinamido)-3-methylbutyl]-5-trifluoromethylbenzene 3c

To a THF (25 mL) solution of 3b (4.20 g, 9.10 mmol) was added trimethylaluminum (2.0 M in toluene, 9.10 mL, 18.2 mmol) at −40° C. and the mixture was stirred for 20 minutes. The mixture was cooled to −78° C. and i-BuLi (1.6 M in heptane from Fluka, 11.4 mL, 18.2 mmol) was added into this mixture and the i-BuLi addition rate was controlled by syringe pump at 1.2 mL/min. After i-BuLi addition, the reaction mixture was stirred for 30 minutes at −78° C., quenched with a 5% aqueous HCl (25 mL) at −78° C., warmed to 10° C. and extracted with EtOAc (3×50 mL). The combined organic layers were washed with brine (30 mL), dried over Na₂SO₄ and evaporated to provide a crude oil which was purified by 1025% EtOAc/Hexanes chromatography to give 4.00 g of 3c as a white foam (85%).

### Step 3D. 2-{4-[3-(2,4-dichlorophenyl)propionyl]-1-piperazinyl}-1-[1S-amino-3-methylbutl]-5-trifluoromethylbenzene 3d

2-[4-(tert-Butoxycarbonyl)-1-piperazinyl]-1-[1S-(S-t-butanesulfinamido)-3-methylbutyl]-5-trifluoromethylbenzene 3c (520 mg, 1 mmol) was allowed to stir at room temperature for 1.5 hours in 20% TFA/DCM mixture. The reaction was quenched with saturated NaHCO₃ solution (5 mL). The organic layer was washed additionally washed with saturated NaHCO₃ solution (2×10 mL), saturated NaCl solution (10 mL), dried over anhydrous MgSO₄, filtered, and solvent removed in vacuo. The deprotected piperazine intermediate was recovered in quantitative yield. A small portion of this piperazine intermediate (52 mg, 0.1 mmol) was dissolved in dichloromethane (0.5 mL) along with HOBt (13.5 mg, 0.1 mmol) and 3-(2,4-dichlorophenyl)propionic acid (25 mg, 0.1 mmol). The reaction mixture was allowed to stir at room temperature for 10 minutes then EDC (19.2 mg, 0.1 mmol) was added. The reaction was then stirred for an additional 8 hours at room temperature followed by quenching with saturated NaHCO₃ solution. The organic layer was separated, washed with saturated NaCl solution (2 mL), dried over anhydrous MgSO₄, filtered, and solvent removed in vacuo. The resulting residue was dissolved in MeOH (2 mL) and 0.2M HCl/ether (1 mL) was added. The reaction was stirred at room temperature for 1 hour then solvent was removed under a stream of nitrogen. The residue was purified by prep HPLC to yield 3d as the TFA salt.

### Step 3E. 2-{4-[3-(2,4-dichlorophenyl)propionyl]-1-piperazinyl}-1-[1S-(3-aminopropionylamido)-3-methylbutyl]-5-trifluoromethylbenzene 3-1

In a 4 dram vial, 2-{4-[3-(2,4-dichlorophenyl)propionyl]-1-piperazinyl}-1-[1S-amino-3-methylbutyl]-5-trifluoromethylbenzene 3d (0.70 g, 1.35 mmol), Boc-p-Alanine (0.2 g, 1.35 mmol), and HOBt (0.183 g, 1.35 mmol) were combined and dissolved in dichloromethane (3 mL). The mixture was capped and stirred for 15 minutes at room temperature. EDC (0.258 g, 1.35 mmol) was added and the mixture was stirred for 1 hour. The mixture was diluted with dichloromethane (2 mL), and was washed with saturated NaHCO₃ solution (2×1 mL) and saturated NaCl solution (1 mL). The organic layer was collected, dried over anhydrous NaSO₄, and filtered. The organic layer was concentrated by a stream of nitrogen. The residue was dissolved in (1:1) TFA/DCM and stirred at room temperature for 30 minutes. Solvent was removed in vacuo. The residue was purified by column chromatography on silica using 1:1 hexane/ethyl acetate as the eluents to afford 3-1 as a light yellow solid in 70% yield. LCMS (tᵣ, 1.301) 587 (M+H).

By the above procedures, the compounds of the following Table 3 were prepared.

TABLE 3

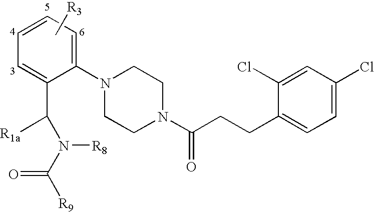

| Cpd | R₃ | R₁ₐ | R₈ | R₉ | (MH⁺) | M.W. | t_{R} |
|---|---|---|---|---|---|---|---|
| 3-1 | 4-CF₃ | (S)-i-Bu | H | —CH₂CH₂NH₂ | 573.1 | 573.48 | 1.250 |
| 3-2 | 4-F | Me | MeOCH₂CH₂— | 2-morpholinyl | 595.2 | 595.54 | 6.582 |
| 3-3 | 4-F | Me | MeOCH₂CH₂— | —CH₂CH₂NH₂ | 553.1 | 553.50 | 6.348 |
| 3-4 | 4-F | Me | MeOCH₂CH₂— | (R)-BnCH(NH₂)CH₂— | 643.2 | 643.63 | 7.338 |
| 3-5 | 4-F | Me | MeOCH₂CH₂— | 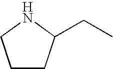 | 593.1 | 593.57 | 6.624 |
| 3-6 | 4-F | Me | MeOCH₂CH₂— | (R)-i-PrCH(NH₂)CH₂— | 595.2 | 595.58 | 6.944 |
| 3-7 | 4-CF₃ | i-Pr | H | —CH₂NH₂ | 559.1 | 559.46 | 7.265 |
| 3-8 | 4-CF₃ | (S)-s-Bu | H | Me | 558.1 | 558.47 | 1.045 |
| 3-9 | 4-CF₃ | (S)-s-Bu | H | —CH₂NH₂ | 573.3 | 573.48 | 7.558 |
| 3-10 | 4-CF₃ | (S)-i-Bu | H | —CH₂NH₂ | 587.1 | 587.51 | 1.308 |
| 3-11 | 4-CF₃ | (S)-s-Bu | H | —CH₂CH₂NH₂ | 587.1 | 587.51 | 2.691 |
| 3-12 | 4-F | EtOCH₂ | H | —CH₂CH₂NH₂ | 539.1 | 539.48 | 1.356 (4) |
| 3-13 | 4-F | BnOCH₂ | H | —CH₂CH₂NH₂ | 601.1 | 601.55 | 1.374 (4) |
| 3-14 | 4-Br | H | NH₂CH₂CH₂— | Me | 557.1 | 556.33 | 6.138 (2) |
| 3-15 | 4-Cl | H | NH₂CH₂CH₂— | Me | 513.0 | 511.88 | 1.613 (4) |

### Example 4

### 1-{4-[3-(4-CHLOROPHENYL)-2-METHYLPROPIONYL]-1-PIPERAZINYL}-2-[1S-(3-AMINOPROPIONYLAMIDO)-3-METHYLBUTYL]-4-TRIFLUOROMETHYLBENZENE

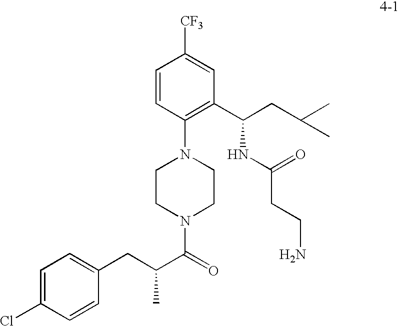

### Step 4A. 2-{4-[3-(2,4-dichlorophenyl)-2-methylpropionyl]-1-piperazinyl}-1-[1S-(S-t-butanesulfinamido)-3-methylbutyl]-5-trifluoromethylbenzene 4a

2-[4-(tert-Butoxycarbonyl)-1-piperazinyl]-1-[1S-(S-t-butanesulfinamido)-3-methylbutyl]-5-trifluoromethylbenzene 3c (520 mg, 1 mmol) was stirred at room temperature for 1.5 hours in 20% TFA/DCM mixture. The reaction was quenched with saturated NaHCO₃ solution (5 mL). The organic layer was washed with saturated NaHCO₃ solution (2×10 mL), saturated NaCl solution (10 mL), was dried over anhydrous MgSO₄, filtered, and the solvent removed in vacuo. The deprotected piperazine intermediate was recovered in quantitative yield. A small portion of this piperazine intermediate (52 mg, 0.1 mmol) was dissolved in dichloromethane (0.5 mL) along with HOBt (13.5 mg, 0.1 mmol) and 3-(4-chlorophenyl)-2-methylpropionic acid (25 mg, 0.1 mmol). The reaction mixture was allowed to stir at room temperature for 10 minutes then EDC (19.2 mg, 0.1 mmol) was added. The reaction was then stirred for an additional 8 hours at room temperature followed by quenching with saturated NaHCO₃ solution. The organic layer was separated, washed with saturated NaCl solution (2 mL), dried over anhydrous MgSO₄, filtered, and the solvent was removed in vacuo. The resulting residue was dissolved in MeOH (2 mL) and 0.2M HCl/ether (1 mL) was added. The reaction was stirred at room temperature for 1 hour then solvent was removed under a stream of nitrogen. The crude product was purified by prep HPLC to yield the compound 4a as the TFA salt.

### Step 4B. 2-{4-[3-(4-chlorophenyl)-2-methylpropionyl]-1-piperazinyl}-1-[1S-(3-aminopropionylamido)-3-methylbutyl]-5-trifluoromethylbenzene 4-1

In a 4 dram vial, 2-{4-[3-(4-chlorophenyl)-2-methylpropionyl]-1-piperazinyl}-1-[1S-(S-t-butanesulfinamido)-3-methylbutyl]-5-trifluoromethylbenzene 4a (0.77 g, 1.24 mmol) was dissolved in methanol (3 mL) and then treated with 2M HCl in ether (4 mL). The mixture was stirred for 45 minutes at room temperature under a nitrogen atmosphere. Solvent was removed in vacuo. The residue was diluted with dichloromethane (3 mL), then was washed with saturated NaHCO₃ (2×2 mL) solution, and saturated NaCl solution (2 mL). The organic layer was collected, dried over anhydrous NaSO₄, and filtered. Solvent was removed in vacuo, to afford 0.70 g of the free base 4b as a light yellow solid. In a 4 dram vial, 2-{4-[3-(4-chlorophenyl)-2-methylpropionyl]-1-piperazinyl}-1-[1S-amino-3-methylbutyl]-5-trifluoromethylbenzene 4b (0.70 g, 1.35 mmol), Boc-p-Alanine (0.2 g, 1.35 mmol), and HOBt (0.183 g, 1.35 mmol) were combined and dissolved in dichloromethane (3 mL). The mixture was capped and stirred for 15 minutes at room temperature. 1-(3-Dimethylaminopropyl) 3-ethylcarbodiimide hydrochloride (EDC) (0.258 g, 1.35 mmol) was added and the mixture was stirred for 1 hour. The mixture was diluted with dichloromethane (2 mL) and was washed with saturated NaHCO₃ solution (2×1 mL) and saturated NaCl solution (1 mL). The organic layer was collected, dried over anhydrous NaSO₄, and filtered. The organic layer was concentrated by a stream of nitrogen. The residue was dissolved in (1:1) TFA/DCM and stirred at room temperature for 30 minutes. Solvent was removed in vacuo and the residue was purified by column chromatography on silica using 1:1 hexane/ethyl acetate as the eluent to afford a light yellow solid 4-lin 70% yield.

By the above procedures, the compounds of the following Table 4 were prepared.

TABLE 4

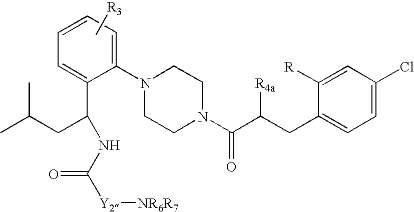

| Cpd | R₃ | —Y₂—NR₆R₇ | R₄ₐ | R | Isomer | (MH⁺) | M.W. | t_{R} |
|---|---|---|---|---|---|---|---|---|
| 4-1 | 4-CF₃ | —CH₂CH₂NH₂ | (R)-Me | H | — | 567.1 | 567.09 | 1.308 (4) |
| 4-2 | 4-CF₃ | —CH₂NH₂ | (R)-Me | H | — | 553.1 | 553.07 | 1.289 (4) |
| 4-3 | 4-CF₃ | —CH₂NHMe | (R)-Me | H | — | 567.1 | 567.09 | 1.257 (4) |
| 4-4 | 4-CF₃ | —CH₂NMe₂ | (R)-Me | H | — | 581.1 | 581.12 | 1.265 (4) |
| 4-5 | 4-CF₃ | (S)-CH(NH₂)Me | (R)-Me | H | — | 567.1 | 567.09 | 1.246 (4) |
| 4-6 | 4-CF₃ | (R)-CH(NH₂)Me | (R)-Me | H | — | 567.1 | 567.09 | 1.241 (4) |
| 4-7 | 4-CF₃ | —C(NH₂)Me₂ | (R)-Me | H | — | 581.1 | 581.12 | 1.287 (4) |
| 4-8 | 4-CF₃ | —CH₂NHMe | Me | —OMe | — | 597.2 | 597.12 | 7.563 (2) |
| 4-9 | 4-CF₃ | —CH₂NMe₂ | Me | —OMe | — | 611.2 | 611.14 | 7.661 (2) |
| 4-10 | 4-CF₃ | —CH₂CH₂NMe₂ | Me | —OMe | — | 625.2 | 625.17 | 11.30 (5) |
| 4-11-I | 4-Cl | —CH₂NH₂ | Me | H | I | 519.1 | 519.51 | 9.840 (1) |
| 4-11-II | 4-Cl | —CH₂NH₂ | Me | H | II | 519.1 | 519.51 | 10.19 (1) |
| 4-12-I | 4-Cl | —CH₂NHMe | Me | H | I | 533.2 | 533.54 | 10.07 (1) |
| 4-12-II | 4-Cl | —CH₂NHMe | Me | H | II | 533.2 | 533.54 | 10.37 (1) |
| 4-13-I | 4-Cl | —CH₂NMe₂ | Me | H | I | 547.2 | 547.57 | 10.27 (1) |
| 4-13-II | 4-Cl | —CH₂NMe₂ | Me | H | II | 547.2 | 547.57 | 10.59 (1) |
| 4-14-I | 4-Cl | —CH₂CH₂NH₂ | Me | H | I | 533.2 | 533.54 | 10.12 (1) |
| 4-14-II | 4-Cl | —CH₂CH₂NH₂ | Me | H | II | 533.2 | 533.54 | 10.27 (1) |
| 4-15-I | 4-Cl | —CH₂CH₂NMe₂ | Me | H | I | 561.2 | 561.59 | 10.24 (1) |
| 4-15-II | 4-Cl | —CH₂CH₂NMe₂ | Me | H | II | 561.2 | 561.59 | 10.54 (1) |
| 4-16 | 4-CF₃ | NH₂CH₂— | (R)-Me | MeO— | — | 583.1 | 583.09 | 1.308 (4) |
| 4-17 | 4-CF₃ | NH₂CH₂CH₂— | (R)-Me | MeO— | — | 597.1 | 597.12 | 1.306 (4) |
| 4-18 | 4-CF₃ | Me₂NCH₂— | (R)-Me | MeO— | — | 611.1 | 611.14 | 1.239 (4) |
| 4-19 | 4-CF₃ | MeNHCH₂— | (R)-Me | MeO— | — | 597.1 | 597.12 | 1.254 (4) |
| 4-20 | 4-CF₃ | (S)-CH(NH₂)Me | (R)-Me | MeO— | — | 597.1 | 597.12 | 1.276 (4) |
| 4-21 | 4-CF₃ | (R)-CH(NH₂)Me | (R)-Me | MeO— | — | 597.1 | 597.12 | 1.268 (4) |
| 4-22 | 4-Cl | MeNHCH₂— | Me | MeO— | — | 563.1 | 563.57 | 10.58 (5) |
| 4-23 | 4-Cl | (S)-CH(NH₂)Me | Me | MeO— | — | 563.1 | 563.57 | 9.500 (5) |
| 4-24 | 4-Cl | 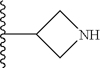 | Me | MeO— | — | 575.1 | 575.58 | 10.60 (5) |
| 4-25 | 4-Cl | Me₂NCH₂CH₂— | Me | MeO— | — | 591.1 | 591.62 | 10.69 (5) |
| 4-26 | 4-Cl | (R)-CH(NH₂)Me | Me | MeO— | — | 563.1 | 563.57 | 10.61 (5) |
| 4-27 | 4-F | NH₂CH₂— | Me | H | — | 503.1 | 503.06 | 1.300 (4) |
| 4-28 | 4-CF₃ | 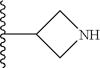 | (R)-Me | MeO— | — | 609.2 | 609.13 | 2.242 (6) |
| 4-29 | 4-Cl | Me₂NCH₂CH₂— | (R)-Me | H | — | 561.2 | 561.59 | 7.126 (2) |
| 4-30 | 4-Me | MeNHCH₂— | (R)-Me | H | — | 513.3 | 513.12 | 9.910 (3) |
| 4-31 | 4-Me | NH₂CH₂CH₂— | (R)-Me | H | — | 513.3 | 513.12 | 9.770 (3) |
| 4-32 | 4-Me | 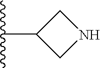 | (R)-Me | H | — | 525.3 | 525.13 | 9.800 (3) |
| 4-33 | 4-Me | 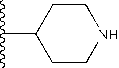 | (R)-Me | H | — | 553.4 | 553.19 | 9.900 (3) |
| 4-34 | 4-Me | 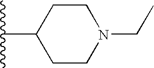 | (R)-Me | H | — | 581.4 | 581.24 | 10.26 (3) |
| 4-35 | 4-Me | 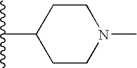 | (R)-Me | H | — | 567.4 | 567.21 | 10.08 (3) |
| 4-36 | 4-Me | 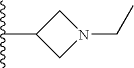 | (R)-Me | H | — | 553.3 | 553.19 | 10.28 (3) |
| 4-37 | 4-Me | 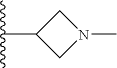 | (R)-Me | H | — | 539.3 | 539.16 | 10.01 (3) |
| 4-38 | 4-Me | Me₂NCH₂— | (R)-Me | H | — | 527.3 | 527.15 | 10.13 (3) |
| 4-39 | 4-Me | MeNHCH₂— | (R)-Me | Me | — | 527.3 | 527.15 | 10.18 (3) |
| 4-40 | 4-Me | MeNHCH₂— | (R)-Me | F | — | 531.2 | 531.11 | 10.14 (3) |
| 4-41 | 4-Me | MeNHCH₂— | (R)-Me | Cl | — | 547.3 | 547.57 | 10.80 (3) |
| 4-42 | 4-Me | MeNHCH₂— | (R)-Me | MeO— | — | 543.3 | 543.15 | 10.14 (3) |
| 4-43 | 4-Me | Me₂NCH₂CH₂— | (R)-Me | Me | — | 554.8 | 555.20 | 1.634 (4) |
| 4-44 | 4-Me | Me₂NCH₂CH₂— | (R)-Me | F | — | 558.8 | 559.17 | 1.562 (4) |
| 4-45 | 4-Me | Me₂NCH₂CH₂— | (R)-Me | Cl | — | 574.7 | 575.62 | 1.636 (4) |
| 4-46 | 4-Me | Me₂NCH₂CH₂— | (R)-Me | MeO— | — | 570.8 | 571.20 | 1.561 (4) |
| 4-47 | 4-Me | NH₂CH₂CH₂— | (R)-Me | Me | — | 531.3 | 530 | 10.58 (3) |
| 4-48 | 4-Me | NH₂CH₂CH₂— | (R)-Me | F | — | 531.3 | 531.11 | 10.52 (3) |
| 4-49 | 4-Me | NH₂CH₂CH₂— | (R)-Me | Cl | — | 547.3 | 547.57 | 11.32 (3) |
| 4-50 | H | Me₂NCH₂CH₂— | (R)-Me | H | — | 527.1 | 527.15 | 5.253 (2) |
| 4-51 | H | Me₂NCH₂CH₂— | (R)-Me | Cl | — | 561.2 | 561.59 | 5.630 (2) |
| 4-52 | H | Me₂NCH₂CH₂— | (R)-Me | F | — | 545.1 | 545.14 | 5.475 (2) |
| 4-53 | H | NH₂CH₂CH₂— | (R)-Me | H | — | 499.3 | 499.10 | 6.235 (2) |
| 4-54 | H | NH₂CH₂CH₂— | (R)-Me | Cl | — | 533.2 | 533.54 | 6.814 (2) |
| 4-55 | H | NH₂CH₂CH₂— | (R)-Me | F | — | 517.0 | 517.09 | 6.880 (2) |
| 4-56 | H | MeNHCH₂— | (R)-Me | H | — | 499.3 | 499.10 | 6.199 (2) |
| 4-57 | H | MeNHCH₂— | (R)-Me | Cl | — | 533.0 | 533.54 | 7.181 (2) |
| 4-58 | H | MeNHCH₂— | (R)-Me | F | — | 517.0 | 517.09 | 6.797 (2) |
| 4-59 | H | MeNHCH₂— | (R)-Me | Me | — | 513.0 | 513.12 | 6.880 (2) |
| 4-60 | H | Me₂NCH₂CH₂— | (R)-Me | Me | — | 541.1 | 541.17 | 5.418 (2) |
| 4-61 | H | NH₂CH₂CH₂— | (R)-Me | Me | — | 513.0 | 513.12 | 6.942 (2) |
| 4-62 | 4-Me | MeNHCH₂CH₂— | (R)-Me | Me | — | 541.2 | 541.17 | 10.53 (3) |
| 4-63 | 4-Me | MeNHCH₂CH₂— | (R)-Me | F | — | 545.2 | 545.14 | 10.47 (3) |
| 4-64 | 4-Me | MeNHCH₂CH₂— | (R)-Me | Cl | — | 562.2 | 561.59 | 11.20 (3) |
| 4-65 | 4-Me | MeNHCH₂CH₂— | (R)-Me | H | — | 527.2 | 527.15 | 10.15 (3) |
| 4-66 | 4-Me | Me₂NCH₂CH₂— | (R)-Me | H | — | 541.2 | 541.17 | 10.20 (3) |
| 4-67 | 6-F | NH₂CH₂CH₂— | (R)-Me | Cl | — | 550.7 | 551.53 | 1.711 (4) |
| 4-68 | 6-F | MeNHCH₂CH₂— | (R)-Me | Cl | — | 564.7 | 565.56 | 1.645 (4) |
| 4-69 | 6-F | Me₂NCH₂CH₂— | (R)-Me | Cl | — | 578.7 | 579.58 | 1.679 (4) |
| 4-70 | 6-F | NH₂CH₂CH₂— | (R)-Me | H | — | 516.8 | 517.09 | 1.776 (4) |
| 4-71 | 6-F | Me₂NCH₂CH₂— | (R)-Me | H | — | 544.8 | 545.14 | 1.667 (4) |
| 4-72 | H | MeNHCH₂CH₂— | (R)-Me | H | — | 513.0 | 513.12 | 5.356 (2) |
| 4-73 | H | MeNHCH₂CH₂— | (R)-Me | Cl | — | 547.0 | 547.57 | 5.549 (2) |
| 4-74 | H | MeNHCH₂CH₂— | (R)-Me | F | — | 531.0 | 531.11 | 5.147 (2) |
| 4-75 | H | MeNHCH₂CH₂— | (R)-Me | Me | — | 527.1 | 527.15 | 5.266 (2) |

### Example 5

### 1-{4-[3-(4-CHLOROPHENYL)-2,2-DIMETHYLPROPIONYL]-1-PIPERAZINYL}-2-[1S-(3-AMINOPROPIONYLAMIDO)-3-METHYLBUTYL]-4-TRIFLUOROMETHYLBENZENE

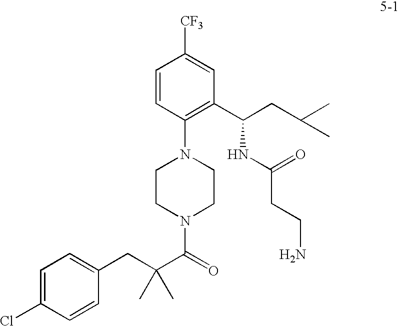

### Step 5A. 2-{4-[3-(4-Chlorophenyl)-2,2-dimethylpropionyl]-1-piperazinyl}-1-[1S-(S-t-butanesulfinamido)-3-methylbutyl]-5-trifluoromethylbenzene 5a

2-[4-(tert-Butoxycarbonyl)-1-piperazinyl]-1-[1S-(S-t-butanesulfinamido)-3-methylbutyl]-5-trifluoromethylbenzene 3c (520 mg, 1 mmol) was stirred at room temperature for 1.5 hours in a 20% TFA/DCM mixture. The reaction was quenched with saturated NaHCO₃ solution (5 mL) and the organic layer was washed with saturated NaHCO₃ solution (2×10 mL) and saturated NaCl solution (10 mL), then was dried over anhydrous MgSO₄, filtered, and solvent removed in vacuo. The deprotected piperazine intermediate was recovered in quantitative yield. A small portion of this piperazine intermediate (52 mg, 0.1 mmol) was dissolved in dichloromethane (0.5 mL) along with HOBt (13.5 mg, 0.1 mmol) and 3-(4-chlorophenyl)-2,2-dimethylpropionic acid (30 mg, 0.1 mmol). The reaction mixture was stirred at room temperature for 10 minutes then EDC (19.2 mg, 0.1 mmol) was added. The reaction was then stirred for an additional 8 hours at room temperature followed by quenching with saturated NaHCO₃ solution. The organic layer was separated, washed with saturated NaCl solution (2 mL), dried over anhydrous MgSO₄, filtered, and solvent removed in vacuo. The resulting residue was dissolved in MeOH (2 mL) and 0.2M HCl/ether (1 mL) was added. The reaction was stirred at room temperature for 1 hour then solvent was removed under a stream of nitrogen. The crude product was purified by prep HPLC to yield the desired product as the TFA salt.

### Step 5B. 2-{4-[3-(4-Chlorophenyl)-2,2-dimethylpropionyl]-1-piperazinyl}-1-[1S-(3-aminopropionylamido)-3-methylbutyl]-5-trifluoromethylbenzene 5-1

In a 4 dram vial, 2-{4-[3-(4-Chlorophenyl)-2,2-dimethylpropionyl]-1-piperazinyl}-1-[1S-amino-3-methylbutyl]-5-trifluoromethylbenzene (0.77 g, 1.24 mmol) was dissolved in methanol (3 mL) and then treated with 2M HCl in ether (4 mL). The mixture was stirred for 45 minutes at room temperature under a nitrogen atmosphere. Solvent was removed in vacuo. The residue was diluted with dichloromethane (3 mL), washed with saturated NaHCO₃ (2×2 mL) solution, and with saturated NaCl solution (2 mL). The organic layer was collected, dried over anhydrous NaSO₄, and filtered. Solvent was removed in vacuo, to afford 0.70 g of the free base 5b as a light yellow solid. In a 4 dram vial, 2-{4-[3-(4-chlorophenyl)-2,2-dimethylpropionyl]-1-piperazinyl}-1-[1S-(3-aminopropionylamido)-3-methylbutyl]-5-trifluoromethylbenzene (0.70 g, 1.35 mmol), Boc-p-Alanine (0.2 g, 1.35 mmol), and HOBt (0.183 g, 1.35 mmol) were combined and dissolved in dichloromethane (3 mL). The mixture was capped and stirred for 15 minutes at room temperature. Then, 1-(3-Dimethylaminopropyl) 3-ethylcarbodiimide hydrochloride (EDC) (0.258 g, 1.35 mmol) was added and the mixture was stirred for 1 hour. The mixture was diluted with dichloromethane (2 mL), washed with saturated NaHCO₃ solution (2×1 mL), and with saturated NaCl solution (1 mL). The organic layer was collected, dried over anhydrous NaSO₄, and filtered. The organic layer was concentrated by a stream of nitrogen. The residue was dissolved in (1:1) TFA/DCM and stirred at room temperature for 30 minutes. Solvent was removed in vacuo. The residue was purified by column chromatography on silica using 1:1 hexane/ethyl acetate as the eluents to afford a light yellow solid in 70% yield.

By the above procedures, the compounds of the following Table 5 were prepared.

TABLE 5

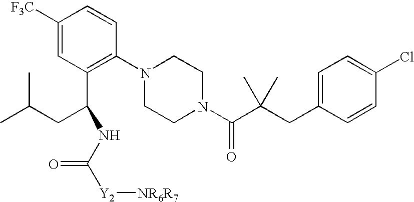

| Cpd | —Y₂—NR₆R₇ | (MH⁺) | MW | t_{R} |
|---|---|---|---|---|
| 5-1 | —CH₂CH₂NH₂ | 581.2 | 581.12 | 7.724 (2) |
| 5-2 | —CH₂NH₂ | 567.2 | 567.09 | 7.660 (2) |
| 5-3 | (S)-CH(NH₂)Me | 581.2 | 581.12 | 7.725 (2) |
| 5-4 | (R)-CH(NH₂)Me | 581.2 | 581.12 | 7.752 (2) |

### Example 6

### 1-{4-[3-(2-METHOXY-4-CHLOROPHENYL)-2R-METHYLPROPIONYL]-1-PIPERAZINYL}-2-{(1S-[2-(DIMETHYLAMINO)ACETAMIDO]-2-METHYLPROPYL}-4-TRIFLUOROMETHYLBENZENE

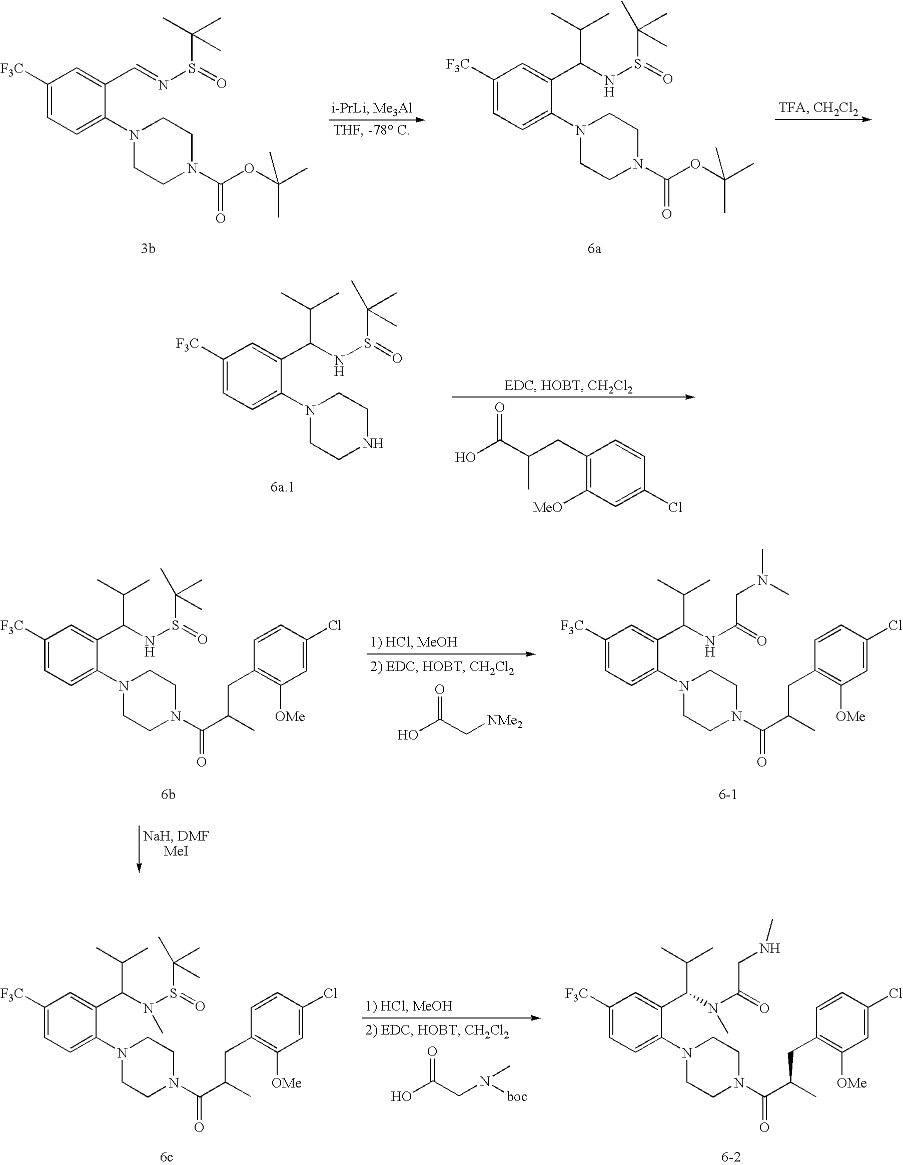

### Step 6A: Compound 6a

To a THF (800 mL) solution of sulfinyl aldimine 3b (103.765 g, 224.7 mmol) was added trimethylaluminum (2.0 M in toluene or heptane, 225 mL, 449.5 mmol) in a 3L 3-neck round-bottom flask at −40° C. and the mixture was stirred for 20 minutes. The mixture was cooled to −78° C. and i-PrLi (0.7 M in pentane from Aldrich, 643 mL, 449.5 mmol) was added into this mixture and the i-PrLi addition rate was controlled by addition funnel to maintain the internal temperature under −70° C. After i-PrLi addition, the reaction mixture was stirred for 10 minutes. The mixture was quenched with 5% aqueous HCl (1400 mL) slowly at −78° C., and when 1000 mL of aq. HCl was added the dry ice-acetone bath was removed and the remaining 400 mL of aq. HCl was added. The mixture was warmed and stirred to −10˜0° C. to ensure that methane gas was released. The organic layer was washed with saturated NaHCO₃ (100 mL) and brine (100 mL) and dried over Na₂SO₄ and evaporated in vacuo to give an orange color foam. The foam was dissolved in petroleum ether (35˜60° C., 50 mL) and crystallized. The solid was collected by filtration, washed with cold hexanes (100 mL×3) to give the 6a as a light yellow solid (42.512 g, 37%). Removing solvents and crystallizing with petroleum ether (30 mL), filtrating and washing gave an additional 19.217 g.

Compound 6a was stirred at room temperature for 1.5 hours in 20% TFA/DCM mixture. The reaction mixture was quenched with saturated NaHCO₃ solution. The organic layer was washed with saturated NaHCO₃ solution and saturated NaCl solution, was dried over anhydrous MgSO₄, filtered, and the solvent removed in vacuo to give 6a.1.

### Step 6B: Compound 6b

To a stirring solution of (R)-3-(4-chloro-2-methoxyphenyl)-2-methyl-propionic acid (1.22 g, 5.36 mmol) and DIEA (1.85 mL, 11.0 mmol) in DMF (27 mL), HBTU (2.64 g, 6.97 mmol) was added in one portion. The mixture was stirred under N₂ for 1 h. A solution of 2-[1-piperazinyl]-1-[1S-(S-t-butanesulfinamido)-2-methylpropyl]-5-trifluoromethylbenzene 6a.1 (2.17 g, 5.36 mmol) in DMF (10 mL) was added and the resulting mixture was stirred at room temperature overnight. The reaction mixture was diluted with EtOAc, washed with 1 N HCl aqueous, sat. NaHCO₃ and brine. The organics were dried (MgSO₄), filtered and evaporated. The residue was purified by column chromatography on silica gel, eluting with a 2:1 to 1:1 v/v mixture of hexane and EtOAc, respectively to give 6b as a foam (1.61 g, 49%).

Compound 6b was dissolved in methanol and then treated with 2M HCl in ether. The mixture was stirred for 45 minutes at room temperature under a nitrogen atmosphere. Solvent was removed in vacuo. The residue was diluted with dichloromethane, then was washed with saturated NaHCO₃ (2×2 mL) solution and saturated NaCl solution (2 mL). The organic layer was collected, dried over anhydrous NaSO₄, and filtered. Solvent was removed in vacuo, to afford deprotected compound 6b.1.

### Step, 6C: Compound 6-1

Dimethylglycine (11 mg, 0.11 mmol) was dissolved in DMF (1 mL) and treated with DIEA (70 μL, 0.40 mmol) and HBTU (63 mg, 0.165 mmol). After 1 h, a solution of (R)-3-(4-Chloro-2-methoxy-phenyl)-2-methyl-1-{4-[2-((S)-2-methyl-1-methylamino-propyl)-4-trifluoromethyl-phenyl]-piperazin-1-yl}-propan-1-one 6b.1 (53 mg, 0.10 mmol) in DMF (0.5 mL) was added and the resulting mixture was stirred overnight at r. t. The reaction was worked-up as usual and the product was purified by preparative HPLC/MS to give 22 mg of compound 6-1.

### Step 6D: Compound 6c

(S)-2-Methyl-propane-2-sulfinic acid [(S)-1-(2-{4-[(R)-3-(4-chloro-2-methoxy-phenyl)-2-methyl-propionyl]-piperazin-1-yl}-5-trifluoromethyl-phenyl)-2-methyl-propyl]-amide 6b (500 mg, 0.813 mmol) was dissolved in DMF (8 mL) under N₂ and cooled to 0° C. with an ice/water bath. NaH (65 mg of a 60% dispersion in oil, 1.63 mmol) was added in one portion. After 20 minutes, MeI (100 μL, 1.63 mmol) was added via syringe and the mixture was stirred at 0° C. for 1.5 h. The reaction was carefully quenched with H₂O and extracted with EtOAc. Evaporation gave compound 6c (506 mg, 98%) which was used in the next step without further purification.

### Step 6E: Compound 6-2

(S)-2-Methyl-propane-2-sulfinic acid [(S)-1-(2-{4-[(R)-3-(4-chloro-2-methoxy-phenyl)-2-methyl-propionyl]-piperazin-1-yl}-5-trifluoromethyl-phenyl)-2-methyl-propyl]-methyl-amide 6c (506 mg, 0.8 mmol) was dissolved in MeOH (8 mL) and treated with HCl (0.3 mL of a 4.0 M solution in dioxane, 1.2 mmol) and stirred at r. t. for 1 h 20 min. The volatiles were removed in vacuo to give (R)-3-(4-chloro-2-methoxy-phenyl)-2-methyl-1-{4-[2-((S)-2-methyl-1-methylamino-propyl)-4-trifluoromethyl-phenyl]-piperazin-1-yl}-propan-1-one which was used in the next step without further purification.

N-boc sarcosine (21 mg, 0.11 mmol) was dissolved in DMF (1 mL) and treated with DIEA (70 μL, 0.40 mmol) and HBTU (63 mg, 0.16 mmol) under N₂. After 30 min., a solution of (R)-3-(4-chloro-2-methoxy-phenyl)-2-methyl-1-{4-[2-((S)-2-methyl-1-methylamino-propyl)-4-trifluoromethyl-phenyl]-piperazin-1-yl}-propan-1-one from the step above (53 mg, 0.10 mg) in DMF (0.5 mL) was introduced via syringe. The resulting mixture was stirred at room temperature overnight. The reaction was diluted with EtOAc, washed with 1 N HCl, sat. NaHCO₃ aqueous and brine. The organics were dried (MgSO₄), filtered and evaporated. The crude residue was dissolved in CH₂Cl₂ (1 mL) and treated with TFA (1 mL) for 1 h. The volatiles were removed in vacuo and the product was purified by preparative HPLC/MS. Compound 6-2 was obtained as the TFA salt. Yield=11.30 mg (0.015 mmol, 16%). LCMS-t_{R} (method 4) 1.475 min. m/z 596.7 (M⁺+H⁺).

By the above procedures, the compounds of the following Table 6 were prepared.

TABLE 6

| Cpd. | —(CR_{1c}R_{1d})ᵣ—NR₆R₇ | R | R₃ | R₄ₐ | R₈ | [MH]⁺ | MW | t_{R} |
|---|---|---|---|---|---|---|---|---|
| 6-1 | Me₂NCH₂— | MeO— | 4-CF₃ | (R)-Me | H | 596.7 | 597.12 | 1.535 (4) |
| 6-2 | MeNHCH₂— | MeO— | 4-CF₃ | (R)-Me | Me | 596.7 | 597.12 | 1.475 (4) |
| 6-3 | Me₂NCH₂— | H | 4-Cl | (R)-Me | Me | 561.7 | 561.59 | 5.341 (2) |
| 6-4 | Me₂NCH₂CH₂— | H | 4-Cl | (R)-Me | H | 546.8 | 547.45 | 1.636 (4) |
| 6-5 | MeNHCH₂— | H | 4-Me | (R)-Me | H | 499.2 | 499.10 | 9.210 (5) |
| 6-6 | H₂NCH₂CH₂— | H | 4-Me | (R)-Me | H | 499.2 | 499.10 | 9.460 (5) |
| 6-7 | MeNHCH₂— | Cl | 6-F | Me | H | 536.7 | 537.50 | 1.638 (4) |
| 6-8 | (R)-CH(NHMe)Me | Cl | 6-F | Me | H | 550.7 | 551.53 | 1.587 (4) |
| 6-9 | 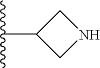 | Cl | 6-F | Me | H | 548.7 | 549.51 | 1.641 (4) |
| 6-10 | Me₂NC(Me)₂— | H | 4-Me | (R)-Me | H | 541.3 | 541.17 | 9.650 (5) |
| 6-11 | Me₂NCH₂CH₂— | H | 4-Me | (R)-Me | H | 527.3 | 527.15 | 9.810 (5) |
| 6-12 | NH₂CH₂— | H | 4-Cl | (R)-Me | H | 505.1 | 505.49 | 1.367 (4) |
| 6-13 | (R)-CH(NH₂)Me | H | 4-Cl | (R)-Me | H | 519.1 | 519.51 | 1.345 (4) |
| 6-14 | (S)-CH(NH₂)Me | H | 4-Cl | (R)-Me | H | 519.1 | 519.51 | 1.348 (4) |
| 6-15 | MeNHCH₂— | H | 4-Cl | (R)-Me | H | 519.1 | 519.51 | 1.374 (4) |
| 6-16 | NH₂CH₂— | MeO— | 4-Cl | (R)-Me | H | 534.7 | 535.51 | 1.587 (4) |
| 6-17 | (R)-CH(NH₂)Me | MeO— | 4-Cl | (R)-Me | H | 548.7 | 549.54 | 1.560 (4) |
| 6-18 | (S)-CH(NH₂)Me | MeO— | 4-Cl | (R)-Me | H | 548.7 | 549.54 | 1.543 (4) |
| 6-19 | MeNHCH₂— | MeO— | 4-Cl | (R)-Me | H | 548.7 | 549.54 | 28.45 (7) |
| 6-20 | —CH(NH₂)i-Pr | MeO— | 4-Cl | (R)-Me | H | 576.7 | 577.59 | 1.530 (4) |
| 6-21 | NH₂CH₂— | H | 6-F | (R)-Me | H | 488.8 | 489.03 | 1.577 (4) |
| 6-22 | (S)-CH(NH₂)Me | H | 6-F | (R)-Me | H | 502.8 | 503.06 | 1.573 (4) |
| 6-23 | (R)-CH(NH₂)Me | H | 6-F | (R)-Me | H | 502.8 | 503.06 | 1.564 (4) |
| 6-24 | MeNHCH₂— | H | 6-F | (R)-Me | H | 502.8 | 503.06 | 1.574 (4) |
| 6-25 | 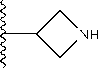 | H | 6-F | (R)-Me | H | 514.8 | 515.07 | 1.585 (4) |
| 6-26 | (S)-CH(NHMe)Me | H | 6-F | (R)-Me | H | 516.8 | 517.09 | 1.526 (4) |
| 6-27 | (R)-CH(NHMe)Me | H | 6-F | (R)-Me | H | 516.8 | 517.09 | 1.508 (4) |
| 6-28 | Me₂NCH₂— | H | 6-F | (R)-Me | H | 516.8 | 517.09 | 1.580 (4) |
| 6-29 | NH₂CH₂— | MeO— | 6-F | (R)-Me | H | 518.7 | 519.06 | 1.579 (4) |
| 6-30 | (S)-CH(NH₂)Me | MeO— | 6-F | (R)-Me | H | 532.7 | 533.08 | 1.560 (4) |
| 6-31 | (R)-CH(NH₂)Me | MeO— | 6-F | (R)-Me | H | 532.7 | 533.08 | 1.553 (4) |
| 6-32 | MeNHCH₂— | MeO— | 6-F | (R)-Me | H | 532.7 | 533.08 | 1.568 (4) |
| 6-33 | 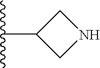 | MeO— | 6-F | (R)-Me | H | 544.7 | 545.10 | 1.608 (4) |
| 6-34 | (S)-CH(NHMe)Me | MeO— | 6-F | (R)-Me | H | 546.7 | 547.11 | 1.539 (4) |
| 6-35 | (R)-CH(NHMe)Me | MeO— | 6-F | (R)-Me | H | 546.8 | 547.11 | 1.531 (4) |
| 6-36 | Me₂NCH₂— | MeO— | 6-F | (R)-Me | H | 546.8 | 547.11 | 1.572 (4) |
| 6-37 | 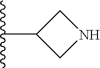 | MeO— | 4-Cl | (R)-Me | H | 560.7 | 561.55 | 1.600 (4) |
| 6-38 | Me- | MeO— | 6-F | (R)-Me | H | 503.8 | 504.04 | 1.418 (4) |
| 6-39 | NH₂CH₂— | H | 4-Me | (R)-Me | H | 484.8 | 485.07 | 1.546 (4) |
| 6-40 | MeNHCH₂— | MeO— | 4-CF₃ | (R)-Me | H | 582.7 | 583.09 | 1.517 (4) |
| 6-41 | 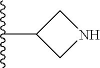 | MeO— | 4-CF₃ | (R)-Me | H | 594.7 | 595.10 | 1.552 (4) |
| 6-42 | Me₂NCH₂CH₂— | H | H | (R)-Me | H | 513 | 513.12 | 5.334 (2) |
| 6-43 | Me₂NCH₂CH₂— | H | 6-F | (R)-Me | H | 530.8 | 531.11 | 1.619 (4) |
| 6-44 | (i-Pr)NHCH₂CH₂— | Cl | 6-F | Me | H | 579.0 | 579.58 | 6.924 |
| 6-45 | (n-Pent)NHCH₂CH₂— | Cl | 6-F | Me | H | 607.0 | 607.64 | 7.733 |
| 6-46 | (i-Bu)NHCH₂CH₂— | Cl | 6-F | Me | H | 593.0 | 593.61 | 7.335 |
| 6-47 | [(3-Me)Bu)NH(CH₂)₂ | Cl | 6-F | Me | H | 607.1 | 607.64 | 7.652 |
| 6-48 | (Bn)NHCH₂CH₂— | Cl | 6-F | (R)-Me | H | 627.0 | 627.63 | 7.547 |
| 6-49 | 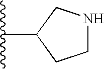 | H | 4-Cl | (R)-Me | H | 545.2 | 545.55 | 9.902 (8) |
| 6-50 | 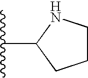 | H | 4-Cl | (R)-Me | H | 545.2 | 545.55 | 10.14 (8) |
| 6-51 | 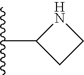 | H | 4-Cl | (R)-Me | H | 531.2 | 531.52 | 9.940 (8) |
| 6-52 | 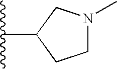 | H | 4-Cl | (R)-Me | H | 559.3 | 559.58 | 10.07 (8) |
| 6-53 | 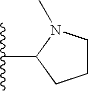 | H | 4-Cl | (R)-Me | H | 559.3 | 559.58 | 10.27 (8) |
| 6-54 | 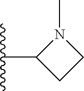 | H | 4-Cl | (R)-Me | H | 545.3 | 545.55 | 10.01 (8) |
| 6-55 | 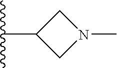 | H | 4-Cl | (R)-Me | H | 545.3 | 545.55 | 9.880 (8) |
| 6-56 | 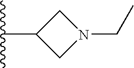 | H | 4-Cl | (R)-Me | H | 559.3 | 559.58 | 10.27 (8) |
| 6-57 | Me₂NCH₂CH₂— | Cl | 4-Cl | Me | H | 581.2 | 582.01 | 7.136 (2) |
| 6-58 | Me₂NCH₂CH₂— | F | 4-Cl | Me | H | 565.2 | 565.56 | 6.781 (2) |
| 6-59 | (i-Pr)NHCH₂CH₂— | H | 4-Cl | (R)-Me | H | 561.2 | 561.59 | 6.748 (2) |
| 6-60 | MeNHCH₂— | Me | 4-Me | (R)-Me | H | 513.3 | 513.12 | 9.790 (3) |
| 6-61 | MeNHCH₂— | F | 4-Me | (R)-Me | H | 517.3 | 517.09 | 9.810 (3) |
| 6-62 | MeNHCH₂— | Cl | 4-Me | (R)-Me | H | 533.3 | 533.54 | 10.36 (3) |
| 6-63 | MeNHCH₂— | MeO— | 4-Me | (R)-Me | H | 529.3 | 529.12 | 9.670 (3) |
| 6-64 | 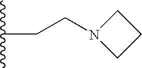 | H | 4-Cl | (R)-Me | H | 559.0 | 559.58 | 5.518 (2) |
| 6-65 | Me₂NCH₂CH₂— | Me | 4-Me | (R)-Me | H | 540.8 | 541.17 | 1.632 (4) |
| 6-66 | Me₂NCH₂CH₂— | F | 4-Me | (R)-Me | H | 544.8 | 545.14 | 1.587 (4) |
| 6-67 | Me₂NCH₂CH₂— | Cl | 4-Me | (R)-Me | H | 560.7 | 561.59 | 1.629 (4) |
| 6-68 | Me₂NCH₂CH₂— | MeO— | 4-Me | (R)-Me | H | 556.8 | 557.17 | 1.638 (4) |
| 6-69 | H₂NCH₂CH₂— | Me | 4-Me | (R)-Me | H | 513.3 | 513.12 | 9.970 (3) |
| 6-70 | H₂NCH₂CH₂— | F | 4-Me | (R)-Me | H | 517.2 | 517.09 | 9.960 (3) |
| 6-71 | H₂NCH₂CH₂— | Cl | 4-Me | (R)-Me | H | 533.2 | 533.54 | 10.65 (3) |
| 6-72 | Me₂NCH₂CH₂— | Cl | H | (R)-Me | H | 547.0 | 547.57 | 6.803 (2) |
| 6-73 | Me₂NCH₂CH₂— | F | H | (R)-Me | H | 531.0 | 531.11 | 6.432 (2) |
| 6-74 | Me₂NCH₂CH₂— | Me | H | (R)-Me | H | 527.0 | 527.15 | 6.527 (2) |
| 6-75 | H₂NCH₂CH₂— | H | H | (R)-Me | H | 485.0 | 485.07 | 6.002 (2) |
| 6-76 | H₂NCH₂CH₂— | Cl | H | (R)-Me | H | 519.0 | 519.51 | 6.302 (2) |
| 6-77 | H₂NCH₂CH₂— | F | H | (R)-Me | H | 503.0 | 503.06 | 6.097 (2) |
| 6-78 | H₂NCH₂CH₂— | Me | H | (R)-Me | H | 499.0 | 499.10 | 6.380 (2) |
| 6-79 | Me₂NCH₂CH₂— | H | H | (R)-Me | H | 513.0 | 513.12 | 6.249 (2) |
| 6-80 | H₂NCH₂CH₂— | Me | 6-F | (R)-Me | H | 516.8 | 517.09 | 1.480 (4) |
| 6-81 | Me₂NCH₂CH₂— | Me | 6-F | (R)-Me | H | 544.8 | 545.14 | 1.428 (4) |
| 6-82 | H₂NCH₂CH₂— | Cl | 6-F | (R)-Me | H | 536.7 | 537.50 | 1.458 (4) |
| 6-83 | Me₂NCH₂CH₂— | Cl | 6-F | (R)-Me | H | 564.7 | 565.56 | 1.394 (4) |
| 6-84 | MeNHCH₂CH₂— | Me | 4-Me | (R)-Me | H | 527.2 | 527.15 | 10.05 (3) |
| 6-85 | MeNHCH₂CH₂— | F | 4-Me | (R)-Me | H | 531.2 | 531.11 | 9.920 (3) |
| 6-86 | MeNHCH₂CH₂— | Cl | 4-Me | (R)-Me | H | 547.2 | 547.57 | 10.62 (3) |
| 6-87 | MeNHCH₂CH₂— | Cl | 6-F | (R)-Me | H | 550.7 | 551.53 | 1.260 (4) |
| 6-88 | MeNHCH₂CH₂— | H | 4-Me | (R)-Me | H | 513.2 | 513.12 | 9.510 (3) |

### Example 7

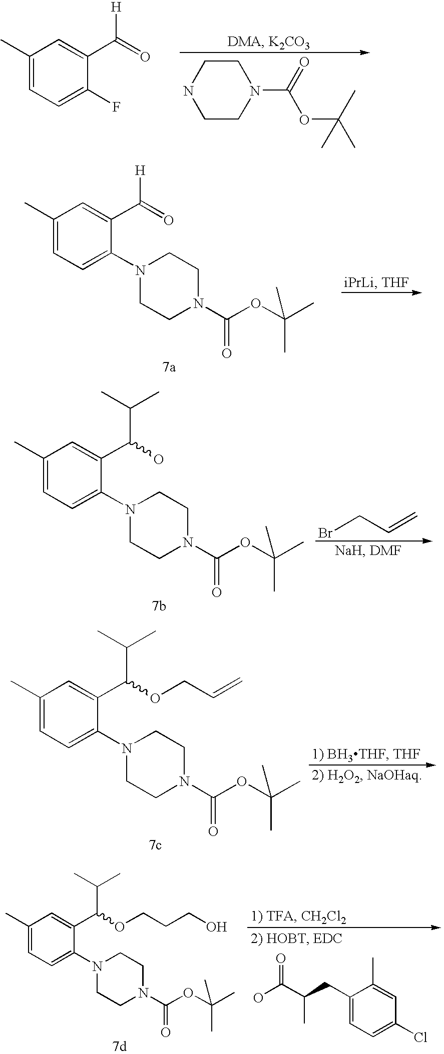

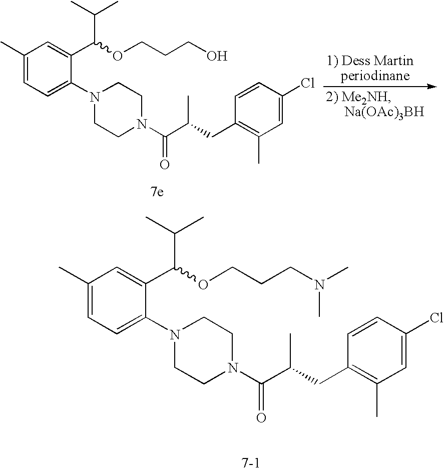

### Step 7A: Compound 7a

To a solution of 2-fluoro-5-methyl-benzaldehyde (50.034 g, 362.2 mmol) and 1-BOC-piperazine (134.926 g, 724.4 mmol) in 100 mL of N,N-dimethylacetamide (DMA) was added K₂CO₃ (125.148 g, 905.5 mmol). The reaction mixture was heated and stirred at 155° C. for 22 hours. The reaction mixture was cooled to room temperature and filtered. The filter was washed with 500 mL of EtOAc. Solvents of the solution were removed in vacuo to give a dark brown tar which was diluted with EtOAc (300 mL). The solution was washed with water (2×150 mL) followed by 5% aqueous HCl (300 mL). The acidic aqueous layer was extracted with EtOAc (250 mL). The combined organic solution was washed with brine (300 mL), dried and the solvent was removed to give a dark yellow solid. Crystallization from EtOAc (˜10-20 mL, washed with 10% EtOAc/Hexanes) gave the 7a as a yellow crystalline solid (48.059 g, 44%). The mother liquor was condensed and dissolved in 30% EtOAc/Hexanes (250 mL) and passed through silica plug (D 50 mm×L 50 mm). The silica plug was washed with 30% EtOAc/Hexanes and evaporation gave a yellow solid which was recrystallized to give 18.706 g of 7a.

### Step 7B: Compound 7b

iso-Propyl Lithium (22 mL of a 0.5 M solution in heptanes, 11.0 mmol) was added dropwise to a stirring solution of 4-(2-formyl-4-methyl-phenyl)-piperazine-1-carboxylic acid tert-butyl ester 7a (3.04 g, 10.0 mmol) in THF (50 mL) at −78° C., under N₂. The resulting dark brown mixture was brought up slowly to room temperature over 1 h. After 2 h at this temperature, the reaction was carefully quenched with H₂O and extracted with EtOAc. The organics were washed with 0.1 N HCl and brine, dried over anhydrous MgSO₄, filtered and evaporated. The crude residue was purified by column chromatography, eluting with a 4:1 v/v mixture of hexanes and EtOAc, respectively. Compound 7b was obtained as a yellow foam (2.07 g, 5.9 mmol, 59%). LCMS-2.426 min. m/z 348.9 (M⁺+H+). ¹H NMR (CDCl₃-300 MHz) δ 0.85 (d, J=6.6 Hz, 3H); 1.01 (d, J=6.6 Hz, 3H); 1.49 (s, 9H); 1.87 (m, J=6.6 Hz, 1H); 2.31 (s, 3H); 2.92-2.86 (m, 4H); 4.47 (t, J=6.9 Hz, 1H); 5.94 (d, J=7.8 Hz, 1H); 6.93 (d, J=1.5 Hz, 1H); 7.05 (dd, J=1.5 Hz, J₂=8.1 Hz, 1H); 7.13 (d, J=8.1 Hz, 1H).

### Step 7C: Compound 7c

Sodium hydride (712 mg of a 60% dispersion in oil, 17.8 mmol) was added portionwise to a stirring solution of 4-[2-(1-hydroxy-2-methyl-propyl)-4-methyl-phenyl]-piperazine-1-carboxylic acid tert-butyl ester 7b (2.07 g, 5.9 mmol) in DMF (30 mL) at 0° C. under N₂. After 40 min., allyl bromide (780 μL, 8.9 mmol) was added dropwise and the mixture allowed to warm to room temperature. After 2 h, the reaction mixture was cooled to 0° C. and quenched carefully with H₂O. The residue was placed in a separatory funnel and extracted with EtOAc. The organics were washed with 0.1 N HCl and brine, dried over anhydrous MgSO₄, filtered and evaporated. The crude residue was purified by column chromatography, eluting with a 95:5 v/v mixture of hexanes and EtOAc, respectively. The 7c was obtained as a thick yellow oil (1.92 g, 4.9 mmol, 83%). LCMS-3.248 min. m/z 388.9 (M⁺+H⁺). ¹H NMR (CDCl₃-300 MHz) δ 0.79 (d, J=6.9 Hz, 3H); 1.01 (d, J=6.9 Hz, 3H); 1.49 (s, 9H); 1.91 (m, J=6.9 Hz, 1H); 2.32 (s, 3H); 2.73-2.66 (m, 2H); 2.88-2.81 (m, 2H); 3.51 (br, 3H); 3.73-3.66 (m, 1H); 3.91-3.85 (m, 1H); 4.65 (d, J=7.2 Hz, 1H); 5.30-5.11 (m, 2H); 5.97-5.84 (m, 1H); 7.05 (s, 2H); 7.21 (s, 1H).

### Step 7D: Compound 7d

A solution of BH₃.THF in THF (10.6 mL of a 1.0 M solution, 10.6 mmol) was added dropwise to a stirring solution of 4-[2-(1-allyloxy-2-methyl-propyl)-4-methyl-phenyl]-piperazine-1-carboxylic acid tert-butyl ester 7c (822 mg, 2.1 mmol) in THF (14 mL) at room temperature, under N₂. The resulting mixture was heated to reflux for 2 hours. The mixture was cooled to room temperature and then to 0° C. (ice/H₂O bath). MeOH (10 mL) was added dropwise to quench the reaction. The volatiles were then removed in vacuo to give an oil, which was immediately dissolved in THF (9 mL), cooled to 0° C. and treated with NaOH (7 mL of a 3 M solution in H₂O, 21.2 mmol) and H₂O₂ (2.4 mL of a 30 vol. Solution, 21.2 mol). The mixture was allowed to slowly reach room temperature and stir overnight. The reaction mix was diluted with EtOAc (100 mL) and placed in a separatory funnel. The organics were washed with H₂O, 0.1 N HCl and brine, dried over anhydrous MgSO₄, filtered and evaporated to give 7d as a thick yellow oil (852 mg, 2.1 mmol, 100%) and it was used in the next step without any further purification. LCMS-2.833 min. m/z 406.9 (M⁺+H⁺).

### Step 7E: Compound 7e

Trifluoroacetic acid (4.2 mL) was added to a stirring solution of 4-{2-[1-(3-hydroxy-propoxy)-2-methyl-propyl]-4-methyl-phenyl}-piperazine-1-carboxylic acid tert-butyl ester 7d (860 mg, 2.1 mmol) in CH₂Cl₂ (21 mL). After 1 hour, the mixture was concentrated in vacuo, and the resulting residue was dissolved in EtOAc (50 mL) and washed with saturated aqueous NaHCO₃. The organics were washed with brine, dried over anhydrous MgSO₄, filtered and evaporated to give a yellow oil (582 mg, 1.9 mmol, 91%) which was used in the next step without further purification. A portion of the resulting free amine (291 mg, 0.95 mmol) was dissolved in CH₂Cl₂ (10 mL) and treated with Et₃N (566 μL, 4.0 mmol), (R)-3-(4-chloro-2-methyl-phenyl)-2-methyl-propionic acid (212 mg, 1.0 mmol) and HOBT (203 mg, 1.5 mmol). The resulting mixture was stirred at room temperature for 30 min. and then was treated with EDC (288 mg, 1.5 mmol). After stirring at room temperature overnight, the reaction mix was diluted with EtOAc (50 mL) and washed with saturated aqueous NaHCO₃ and 0.1 N HCl. The organics were washed with brine, dried over anhydrous MgSO₄, filtered and evaporated. The residue was dissolved in THF (3 mL) and treated with NaOH (3 mL of a 1 N aqueous solution). The mixture was refluxed for 24 h. After cooling, the mix was diluted with EtOAc (50 mL) and washed with saturated aqueous NaHCO₃ and 0.1 N HCl. The organics were washed with brine, dried over anhydrous MgSO₄, filtered and evaporated. The residue was purified by column chromatography, eluting with a 1:1 v/v mixture of hexanes and EtOAc, respectively to give 7e as a white foam (95 mg, 0.19 mmol, 20%). LCMS-2.933 min. m/z 500.8 (M⁺+H⁺).

### Step 7F: Compound 7-1

(R)-3-(4-Chloro-2-methyl-phenyl)-1-(4-{2-[1-(3-hydroxy-propoxy)-2-methyl-propyl]-4-methyl-phenyl}-piperazin-1-yl)-2-methyl-propan-1-one 7e (95 mg, 0.19 mmol) was dissolved in CH₂Cl₂ (1.9 mL) and treated with Dess-Martin periodinane (121 mg, 0.29 mmol) and H₂O (1 drop). A milky suspension resulted. The mixture was stirred at room temperature for 1.5 h, and then was diluted with Et₂O and treated with a 1:1 v/v mixture of 10% aqueous Na₂S₂O₃ and saturated aqueous NaHCO₃. After stirring for 10 min., the mixture was transferred to a separatory funnel and the organics were separated, dried over anhydrous MgSO₄, filtered and evaporated to give a colorless film (80 mg, 0.16 mmol, 85%). Part of this material (40 mg, 0.08 mmol) was dissolved in CH₂Cl₂ (1 mL) and was treated with dimethylamine (120 μL of a 2.0 M solution in THF, 0.24 mmol). After 30 min., Na(OAc)₃ BH (51 mg, 0.24 mmol) was added and the resulting mixture was stirred at room temperature for 2 h. The volatiles were removed in vacuo and the residue was dissolved in EtOAc (10 mL). This was washed with saturated aqueous NaHCO₃, brine, dried over anhydrous MgSO₄, filtered and evaporated. The residue was purified by preparative HPLC/MS to give the 7-1 as the TFA salt. LCMS-1.379 min. m/z 528.1 (M⁺+H⁺).

By the above procedures, the compounds of the following Table 7 were prepared.

TABLE 7

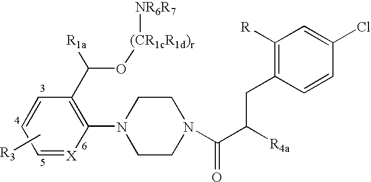

| Cpd | X | —(CR_{1c}R_{1d})ᵣ—NR₆R₇ | R₃ | R₁ₐ | R | R₄ₐ | [M+H]⁺ | MW | t_{R} |
|---|---|---|---|---|---|---|---|---|---|
| 7-1 | CH | —CH₂CH₂CH₂NMe₂ | 4-Me | i-Pr— | Me | Me | 528.1 | 528.18 | 1.379 |
| 7-2 | CH | —CH₂CH₂CH₂NHMe | 4-Me | i-Pr— | Me | Me | 514.1 | 514.15 | 1.444 |

### Example 8

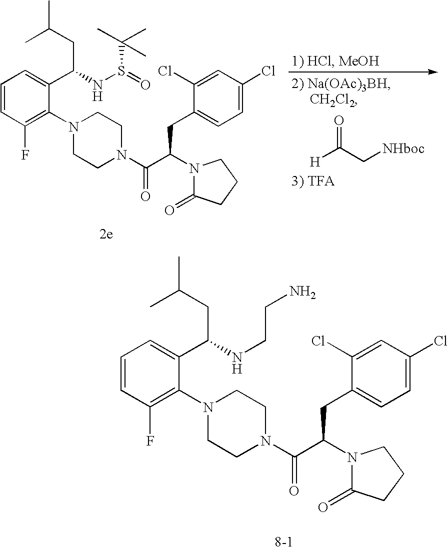

### Step 8A: Compound 8-1

A stirring solution of 2-{4-[3-(2,4-dichlorophenyl)-2R-(2-oxo-1-pyrrolidinyl)-1-piperazinyl]-3-fluoro-1-[1S-(S-t-butanesulfinamido)-3-methylbutyl]benzene 2e (458 mg, 0.70 mmol) in MeOH (7 mL) was treated with HCl (350 μL of a 4.0 M solution in dioxane, 1.40 mmol) and the resulting mixture was stirred at room temperature for 1.5 h. The mixture was concentrated in vacuo and the crude residue was dissolved in CH₂Cl₂ (14 mL) and was treated with (2-oxo-ethyl)-carbamic acid tert-butyl ester (167 mg, 1.0 mmol) and 1 drop of HOAc. After 15 min., Na(OAc)₃BH (446 mg, 2.10 mmol) was added portionwise. The resulting mixture was stirred at room temperature overnight. The mixture was quenched by the addition of saturated aqueous NH₄Cl. The reaction mix was diluted with EtOAc (100 mL) and placed in a separatory funnel. The organic layer was washed with saturated aqueous NaHCO₃ and brine, dried over anhydrous MgSO₄, filtered and evaporated. The residue was dissolved in CH₂Cl₂ (8 mL) and treated with TFA (8 mL) for 1 h. The volatiles were removed in vacuo and the crude residue was purified by preparative HPLC/MS to give the 8-1 as the TFA salt (150 mg, 0.25 mmol, 36% yield). LCMS-2.326 min. m/z 592.2 (M⁺+H⁺).

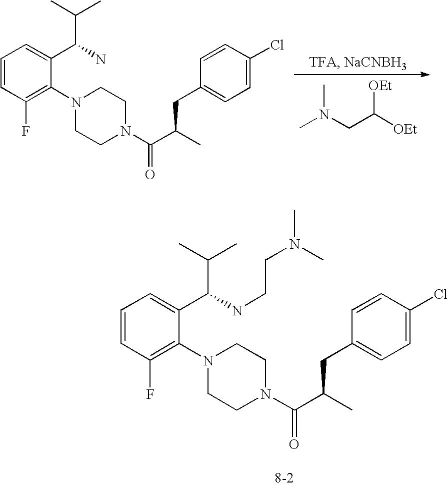

### Step 8B: Compound 8-2

To a flask containing (R)-1-{4-[2-((S)-1-amino-2-methyl-propyl)-6-fluoro-phenyl]-piperazin-1-yl}-3-(4-chloro-phenyl)-2-methyl-propan-1-one (43 mg, 0.10 mmol) and (2,2-diethoxy-ethyl)-dimethyl-amine (100 μL, 0.50 mmol), TFA (300 μL, 4.0 mmol) was added via syringe. After 5 min., NaCNBH₃ (95 mg, 1.5 mmol) was added and the resulting mixture was heated to 120° C. overnight. The resulting brown reaction mixture was concentrated in vacuo and purified via preparative HPLC/MS to furnish 8-2 (3.8 mg, 0.006 mmol, 6%) as the TFA salt. LCMS-t_{R} (method 4) 1.596 min. m/z 502.8 (M⁺+H⁺).

By the above procedures, the compounds of the following Tables 8A and 8B were prepared.

TABLE 8A

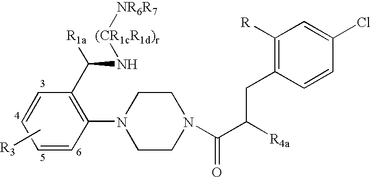

| Cpd | —(CR_{1c}R_{1d})ᵣ—NR₆R₇ | R₃ | R₁ₐ | R | R₄ₐ | [M + H]⁺ | MW | t_{R} |
|---|---|---|---|---|---|---|---|---|
| 8-1 | —CH₂CH₂NH₂ | 6-F | i-Bu | Cl | 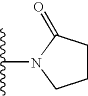 | 592.1 | 592.58 | 7.98 |
| 8-2 | —CH₂CH₂NMe₂ | 6-F | i-Pr | H | Me | 502.8 | 503.10 | 1.596 |
| 8-3 | —CH₂CH₂NH₂ | 4-CF₃ | i-Bu | H | Me | 539.1 | 539.08 | 1.400 |
| 8-4 | —CH₂CH₂CH₂NH₂ | 4-CF₃ | i-Bu | MeO— | Me | 583.2 | 583.13 | 6.355 |
| 8-5 | —CH₂CH₂NH₂ | 4-Cl | i-Pr | MeO— | Me | 520.7 | 521.53 | 1.715 |
| 8-6 | —CH₂CH₂NH₂ | 6-F | i-Pr | H | Me | 474.8 | 475.05 | 1.750 |
| 8-7 | —CH₂CH₂NHMe | 6-F | i-Pr | H | Me | 488.7 | 489.07 | 1.713 |
| 8-8 | —CH₂CH₂NH₂ | 6-F | i-Pr | MeO— | Me | 504.8 | 505.07 | 1.730 |
| 8-9 | —CH₂CH₂NHMe | 4-Cl | i-Bu | MeO— | Me | 549.2 | 549.58 | 8.66 |
| 8-10 | —CH₂CH₂NMe₂ | 4-Cl | i-Pr | MeO— | Me | 548.8 | 549.58 | 1.234 |
| 8-11 | —CH₂CH₂NMe₂ | 6-F | i-Pr | MeO— | Me | 532.8 | 533.13 | 1.623 |
| 8-12 | —CH₂CH₂NH₂ | 4-CF₃ | i-Pr | MeO— | Me | 554.8 | 555.08 | 1.657 |
| 8-13 | —CH₂CH₂NHMe | 4-Me | i-Pr | H | Me | 485.2 | 485.11 | 7.65 |
| 8-14 | 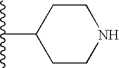 | 4-Me | i-Bu | H | Me | 525.3 | 525.18 | 7.71 |
| 8-15 | —CH₂CH₂NHMe | 4-Me | i-Bu | H | Me | 499.3 | 499.14 | 8.20 |
| 8-16 | —CH₂CH₂NMe₂ | 4-Me | i-Pr | Me | Me | 513.3 | 513.17 | 8.01 |
| 8-17 | —CH₂CH₂NMe₂ | 4-Me | i-Pr | F | Me | 517.3 | 517.13 | 8.01 |
| 8-18 | —CH₂CH₂NMe₂ | 4-Me | i-Pr | Cl | Me | 533.3 | 533.58 | 8.47 |
| 8-19 | —CH₂CH₂NMe₂ | 4-Me | i-Bu | Me | Me | 527.3 | 527.19 | 8.53 |
| 8-20 | —CH₂CH₂NMe₂ | 4-Me | i-Bu | F | Me | 531.3 | 531.16 | 8.47 |
| 8-21 | —CH₂CH₂NMe₂ | 4-Me | i-Bu | Cl | Me | 547.3 | 547.61 | 9.01 |
| 8-22 | —CH₂CH₂NHMe | 6-F | i-Pr | Me | Me | 502.8 | 503.10 | 1.614 |
| 8-23 | —CH₂CH₂NHMe | 6-F | i-Pr | Cl | Me | 523.0 | 523.52 | 1.864 |
| 8-24 | —CH₂CH₂NHMe | H | i-Bu | H | Me | 485.0 | 485.11 | 5.249 |
| 8-25 | —CH₂CH₂NHMe | H | i-Bu | F | Me | 503.0 | 503.10 | 5.409 |
| 8-26 | —CH₂CH₂CH₂NH₂ | H | i-Bu | H | Me | 485.0 | 485.11 | 4.629 |
| 8-27 | —CH₂CH₂CH₂NH₂ | H | i-Bu | Cl | Me | 519.0 | 519.56 | 4.810 |
| 8-28 | —CH₂CH₂CH₂NH₂ | H | i-Bu | F | Me | 503.0 | 503.1 | 4.725 |
| 8-29 | —CH₂CH₂CH₂NH₂ | H | i-Bu | Me | Me | 499.1 | 499.14 | 4.488 |
| 8-30 | —CH₂CH₂NHMe | 4-Me | i-Pr | Me | Me | 499.2 | 499.14 | 5.315 |
| 8-31 | —CH₂CH₂NHMe | 4-Me | i-Pr | F | Me | 503.3 | 503.1 | 5.141 |
| 8-32 | —CH₂CH₂NHMe | 4-Me | i-Bu | F | Me | 517.3 | 517.13 | 5.529 |
| 8-33 | —CH₂CH₂NHMe | 4-Me | i-Bu | Cl | Me | 533.3 | 533.58 | 5.818 |
| 8-34 | —CH₂CH₂NH₂ | 4-CF₃ | i-Bu | Cl | 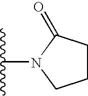 | 642.1 | 642.59 | 8.36 |
| 8-35 | —CH₂CH₂CH₂NH₂ | 6-F | i-Bu | Cl | 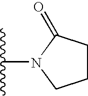 | 606.1 | 606.61 | 7.70 |
| 8-36 | —CH₂CH₂CH₂NH₂ | 4-CF₃ | i-Bu | Cl | 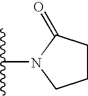 | 656.2 | 656.62 | 8.44 |
| 8-37 | —CH₂CH₂NH₂ | 4-F | i-Bu | Cl | 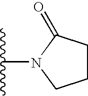 | 592.1 | 592.58 | 1.378 |
| 8-38 | —CH₂CH₂NHMe | 4-F | i-Bu | Cl | 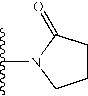 | 606.1 | 606.61 | 1.370 |
| 8-39 | —CH₂CH₂NMe₂ | 4-F | i-Bu | Cl | 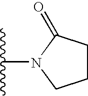 | 620.1 | 620.64 | 1.329 |
| 8-40 | —CH₂CH₂NEt₂ | 4-F | i-Bu | Cl | 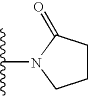 | 648.1 | 648.69 | 1.286 |
| 8-41 | —CH₂CH₂NHMe | 6-F | i-Pr | H | 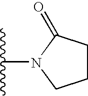 | 558.3 | 558.14 | 6.68 |
| 8-42 | —CH₂CH₂NHMe | 6-F | i-Pr | Cl | 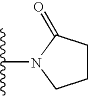 | 592.2 | 592.58 | 7.11 |
| 8-43 | —CH₂CH₂NH₂ | 6-F | i-Pr | Cl | 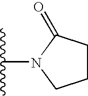 | 578.2 | 578.56 | 7.30 |
| 8-44 | —CH₂CH₂NMe₂ | 4-CF₃ | i-Bu | H | 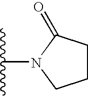 | 635.8 | 636.20 | 1.515 |
| 8-45 | —CH₂CH₂NH₂ | 4-CF₃ | i-Bu | H | 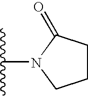 | 607.8 | 608.14 | 1.558 |
| 8-46 | —CH₂CH₂NMe₂ | 6-F | i-Bu | H | 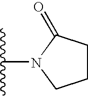 | 585.8 | 586.19 | 1.669 |
| 8-47 | —CH₂CH₂NHMe | 6-F | i-Bu | H | 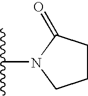 | 571.8 | 572.16 | 1.704 |
| 8-48 | —CH₂CH₂NH₂ | 6-F | i-Bu | H | 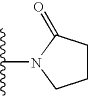 | 557.8 | 558.14 | 1.600 |
| 8-49 | —CH₂CH₂NH₂ | 6-F | i-Pr | H | 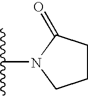 | 543.8 | 544.11 | 1.638 |
| 8-50 | —CH₂CH₂NMe₂ | 6-F | i-Pr | H | 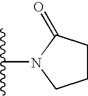 | 572.3 | 572.16 | 7.17 |
| 8-51 | —CH₂CH₂NMe₂ | 6-F | i-Pr | Cl | 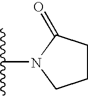 | 606.2 | 606.61 | 7.74 |
| 8-52 | —CH₂CH₂NHMe | H | i-Bu | H | 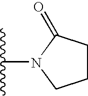 | 554.0 | 554.17 | 5.047 |
| 8-53 | —CH₂CH₂NHMe | H | i-Bu | Cl | 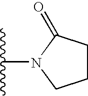 | 588.0 | 588.62 | 5.299 |
| 8-54 | —CH₂CH₂CH₂NH₂ | H | i-Bu | H | 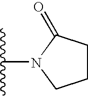 | 554 | 544.17 | 4.357 |
| 8-55 | —CH₂CH₂CH₂NH₂ | H | i-Bu | Cl | 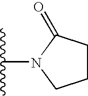 | 588.1 | 588.62 | 4.606 |

TABLE 8B

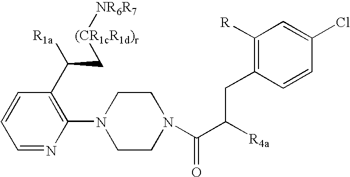

| Cpd. | —(CR_{1c}R_{1d})ᵣ—NR₆R₇ | R₃ | R₁ₐ | R | R₄ₐ | [M + H]⁺ | MW | t_{R} |
|---|---|---|---|---|---|---|---|---|
| 8-56 | —CH₂CH₂NNHMe | H | i-Bu | Cl | Me | 520.2 | 520.54 | 7.90 |

### Example 9

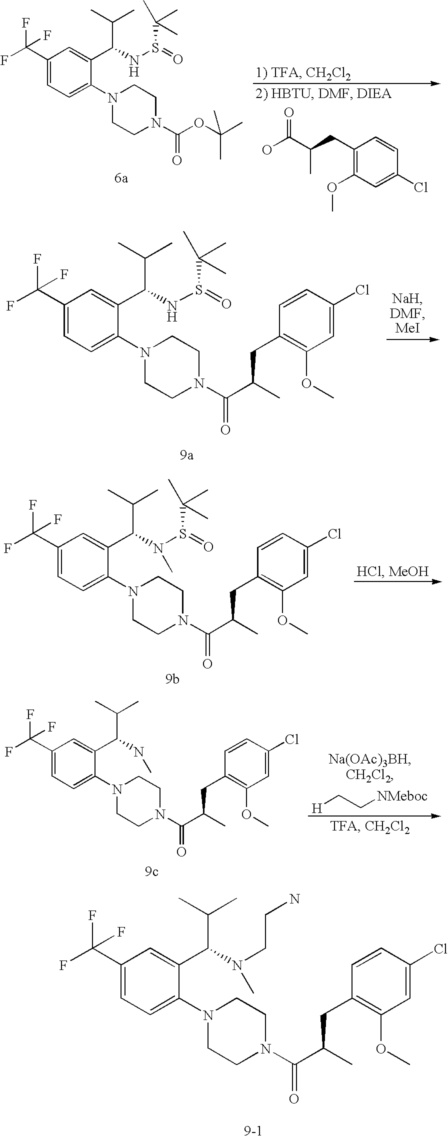

### Step 9A: Compound 9a

4-{2-[(S)-2-Methyl-1-((S)-2-methyl-propane-2-sulfinylamino)-propyl]-4-trifluoromethyl-phenyl}-piperazine-1-carboxylic acid tert-butyl ester 6a (3.80 g, 7.52 mmol) was dissolved in CH₂Cl₂ (75 mL) and treated with TFA (15 mL) at room temperature. The resulting solution was stirred for 1.5 h, after which time the mixture was poured onto saturated aqueous NaHCO₃ slowly in order to neutralize the TFA. CH₂Cl₂ (100 mL) was added and the organic layer was separated, dried over anhydrous MgSO₄, filtered and evaporated to give 2.28 g (5.63 mmol, 75%) of deprotected piperazine compound (S,S)-2-methyl-propane-2-sulfinic acid [2-methyl-1-(2-piperazin-1-yl-5-trifluoromethyl-phenyl)-propyl]-amide.

To a stirring solution of (R)-3-(4-Chloro-2-methoxy-phenyl)-2-methyl-propionic acid (1.22 g, 5.36 mmol) and DIEA (1.85 mL, 11.0 mmol) in DMF (27 mL), HBTU (2.64 g, 6.97 mmol) was added in one portion. The mixture was stirred under N₂ for 1 h. A solution of (S,S)-2-methyl-propane-2-sulfinic acid [2-methyl-1-(2-piperazin-1-yl-5-trifluoromethyl-phenyl)-propyl]-amide obtained above (2.17 g, 5.36 mmol) in DMF (10 mL) was added and the resulting mixture was stirred at room temperature overnight. The mixture was diluted with EtOAc, washed with 1 N HCl aqueous, sat. NaHCO₃ aqueous and brine. The organics were dried (MgSO₄), filtered and evaporated. The residue was purified by column chromatography on silica gel, eluting with a 2:1 to 1:1 v/v mixture of hexane and EtOAc, respectively to give 9a as a foam (1.61g, 49%).

### Step 9B: Compound 9b

(S)-2-Methyl-propane-2-sulfinic acid [(S)-1-(2-{4-[(R)-3-(4-chloro-2-methoxy-phenyl)-2-methyl-propionyl]-piperazin-1-yl}-5-trifluoromethyl-phenyl)-2-methyl-propyl]-amide 9a (500 mg, 0.813 mmol) was dissolved in DMF (8 mL) under N₂ and cooled to 0° C. with an ice/water bath. NaH (65 mg of a 60% dispersion in oil, 1.63 mmol) was added in one portion. After 20 minutes, MeI (100 μL, 1.63 mmol) was added via syringe and the reaction was stirred at 0° C. for 1.5 h. The reaction was carefully quenched with H₂O and extracted with EtOAc. Compound 9b (506 mg, 98%) was used in the next step without further purification.

### Step 9C: Compound 9c

(S)-2-Methyl-propane-2-sulfinic acid [(S)-1-(2-{4-[(R)-3-(4-chloro-2-methoxy-phenyl)-2-methyl-propionyl]-piperazin-1-yl}-5-trifluoromethyl-phenyl)-2-methyl-propyl]-methyl-amide 9b (506 mg, 0.8 mmol) was dissolved in MeOH (8 mL) and treated with HCl (0.3 mL of a 4.0 M solution in dioxane, 1.2 mmol) and stirred at room temperature for 1 h 20 min. The volatiles were removed in vacuo to give 9c which was used in the next step without further purification.

### Step 9D: Compound 9-1

(R)-3-(4-Chloro-2-methoxy-phenyl)-2-methyl-1-{4-[2-((S)-2-methyl-1-methylamino-propyl)-4-trifluoromethyl-phenyl]-piperazin-1-yl}-propan-1-one hydrochloride salt 9c from the step above (105 mg, 0.20 mmol) was dissolved in CH₂Cl₂ (2 mL) and treated with (2-oxo-ethyl)-carbamic acid tert-butyl ester (63 mg, 0.40 mmol) and a drop of HOAc. Na(OAc)₃BH (170 mg, 0.80 mg) was added and the resulting mixture was stirred at ambient temperature overnight. The reaction was quenched with saturated aqueous NaHCO₃. CH₂Cl₂ (20 mL) was added and the organic layer was separated, dried over anhydrous MgSO₄, filtered and evaporated. A 1:1 v/v mixture of CH₂Cl₂ and TFA (2 mL) was then introduced. After stirring for 2 h, the volatiles were removed under reduced pressure and the residue was purified by preparative HPLC/MS to afford 9-1 (19.2 mg (0.03 mmol, 14%) as the TFA salt. LCMS-1.520 min. m/z 568.8 (M⁺+H⁺).

By the above procedures, the compounds of the following Table 9 were prepared.

TABLE 9

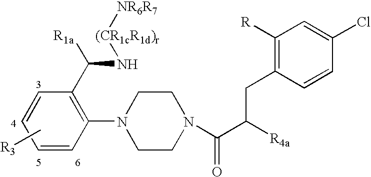

| | —(CR_{1c}R_{1d})ᵣ— | | | | | | | |
|---|---|---|---|---|---|---|---|---|
| Cpd. | NR₆R₇ | R₃ | R₁ₐ | R | R₄ₐ | [M + H]⁺ | MW | t_{R} |
| 9-1 | —CH₂CH₂NH₂ | 4- | i-Pr | MeO— | Me | 568.8 | 569.11 | 1.52 |
| | | CF₃ | | | | | | |
| 9-2 | —CH₂CH₂NHMe | 6-F | i-Pr | H | Me | 502.8 | 503.1 | 1.617 |
| 9-3 | —CH₂CH₂NH₂ | 6-F | i-Bu | Cl | 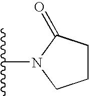 | 605.7 | 606.61 | 5.478 |

### Example 10

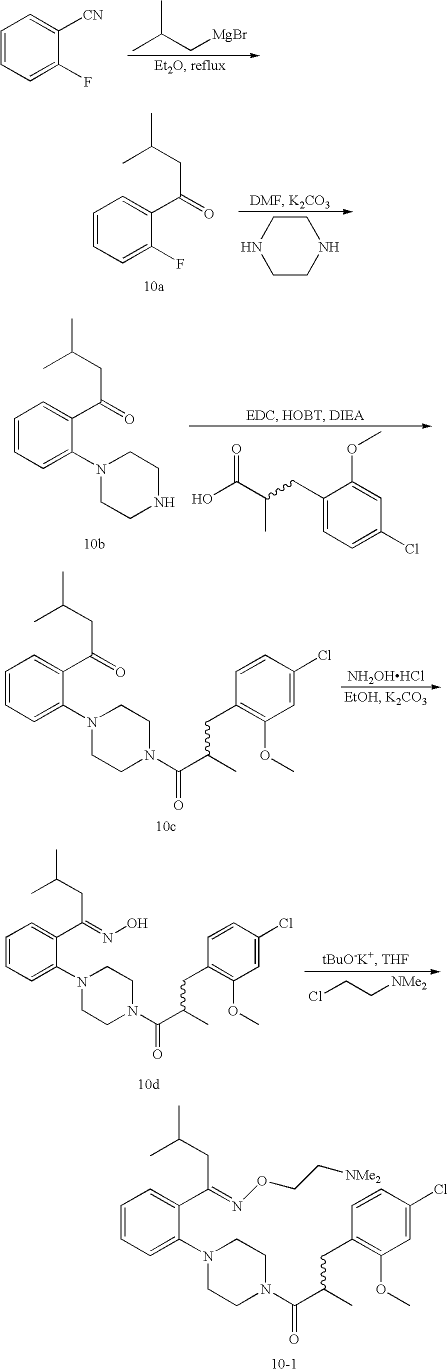

### Step 10A: Compound 10a

Isobutyl magnesium bromide (60 mL of a 2.0 M solution in THF, 120 mmol) was added dropwise to a stirring solution of 2-fluorobenzonitrile (12.11 g, 100 mmol) in THF (40 mL) at 0° C. The resulting solution was slowly warmed to room temperature and was stirred at that temperature for 4 h. Cooling to 0° C. was followed by quenching with aqueous saturated NH₄Cl. The mixture was extracted with EtOAc, washed with brine, dried over anhydrous MgSO₄, filtered and evaporated to give 10a as a yellow oil (14.5 g, 80.6 mmol, 81%).

### Step 10B: Compound 10b

A suspension containing 1-(2-fluoro-phenyl)-3-methyl-butan-1-one 10a (5.41 g, 30 mmol), DMF (50 mL), piperazine (12.92 g, 150 mmol) and K₂CO₃ (8.29 g, 60 mmol) was heated at 120° C. for 16 h. After cooling to room temperature, the reaction mix was diluted with H₂O and extracted with EtOAc. The organic layer was washed with brine, dried over anhydrous MgSO₄, filtered and evaporated. The residue was purified by column chromatography, eluting with a 9:1 v/v mixture of CH₂Cl₂ and MeOH, respectively to give 10b (3.48 g, 14.15 mmol, 47%) as a pale yellow solid. LCMS m/z 247.1 [M⁺+1].

### Step 10C: Compound 10c

To a stirring solution of 3-methyl-1-(2-piperazin-1-yl-phenyl)-butan-1-one 10b (1.23 g, 5.0 mmol), 3-(4-chloro-2-methoxy-phenyl)-2-methyl-propionic acid (1.26 g, 5.5 mmol) and Hünig's base (1.74 mL, 10.0 mmol) in CH₂Cl₂ (50 mL), HOBT (1.01 g, 7.5 mmol) was added. After 30 min. at room temperature, EDC (1.44 g, 7.5 mmol) was introduced. The resulting mixture was stirred at room temperature overnight. The reaction mixture was diluted with EtOAc and washed with 0.1 N HCl, saturated NaHCO₃ and brine. The organic layer was dried over anhydrous MgSO₄, filtered and evaporated. The residue was purified by column chromatography, eluting with a 4:1 v/v mixture of hexanes and EtOAc, respectively. Compound 10c (1.22 g, 2.68 mmol, 54%) was obtained as a yellow foam. LCMS-2.593 min. m/z 457.0 [M⁺+1]. ¹H NMR (CDCl₃-300 MHz) δ 0.93 (d, J=6.3 Hz, 6H); 1.13 (d, J=6.6 Hz, 3H); 2.11 (m, 1H); 2.38-2.30(m, 1H); 2.88-2.65(m, 7H); 3.14-3.07(m, 1H); 3.48 (m, 3H); 3.81 (s, 3H); 3.93-3.88 (m, 1H); 6.88-6.82 (m, 2H); 7.05-6.96 (m, 2H); 7.14-7.09 (m, 1H); 7.31-7.28 (m, 1H); 7.42-7.37 (m, 1H).

### Step 10D: Compound 10d

A suspension of 1-(2-{4-[3-(4-chloro-2-methoxy-phenyl)-2-methyl-propionyl]-piperazin-1-yl}-phenyl)-3-methyl-butan-1-one 10c (456 mg, 1.0 mmol), hydroxylamine hydrochloride (140 mg, 2.0 mmol) and K₂CO₃ (331 mg, 2.4 mmol) in EtOH (10 mL) was refluxed for 3.5 h. The reaction mixture was cooled to room temperature, diluted with EtOAc and washed with H₂O and brine. After drying over anhydrous MgSO₄, the mixture was filtered and evaporated to give 10d (375 mg, 0.80 mmol, 80%) as a mixture of cis and trans isomers. Yield=. LCMS-2.246 and 2.341 min. (cis and trans) m/z 472.0 [M⁺+1].

### Step 10E: Compound 10-1

1-(2-{4-[3-(4-Chloro-2-methoxy-phenyl)-2-methyl-propionyl]-piperazin-1-yl}-phenyl)-3-methyl-butan-1-one oxime 10d (100 mg, 0.21 mmol) was dissolved in THF (2.1 mL) under N₂. Potassium tert-butoxide (54 mg, 0.46 mmol) was added and the mixture was stirred at room temperature for 30 min. (2-Chloro-ethyl)-dimethyl-amine hydrochloride (36 mg, 0.25 mmol) was added and the mixture was kept at room temperature for 2 h. LCMS indicated only 5% conversion to the product. The mixture was treated with additional amounts of potassium tert-butoxide (90 mg) and (2-chloro-ethyl)-dimethyl-amine hydrochloride (40 mg) and then heated to reflux. After 2 h, approximately 50% had been converted to product. The reaction was cooled, diluted with H₂O and extracted with EtOAc. The organic layer was washed with brine, dried over anhydrous MgSO₄, filtered and evaporated. The residue was purified by preparative TLC plate, eluting with a 400:50:2 v/v mixture of CHCl₃, MeOH and NH₄OH, respectively. Compound 10-1 (77 mg, 0.14 mmol, 68%) was obtained as a mixture of cis and trans isomers. LCMS-5.148 min. (cis and trans) m/z 543.0 [M⁺+1].

### Example 11

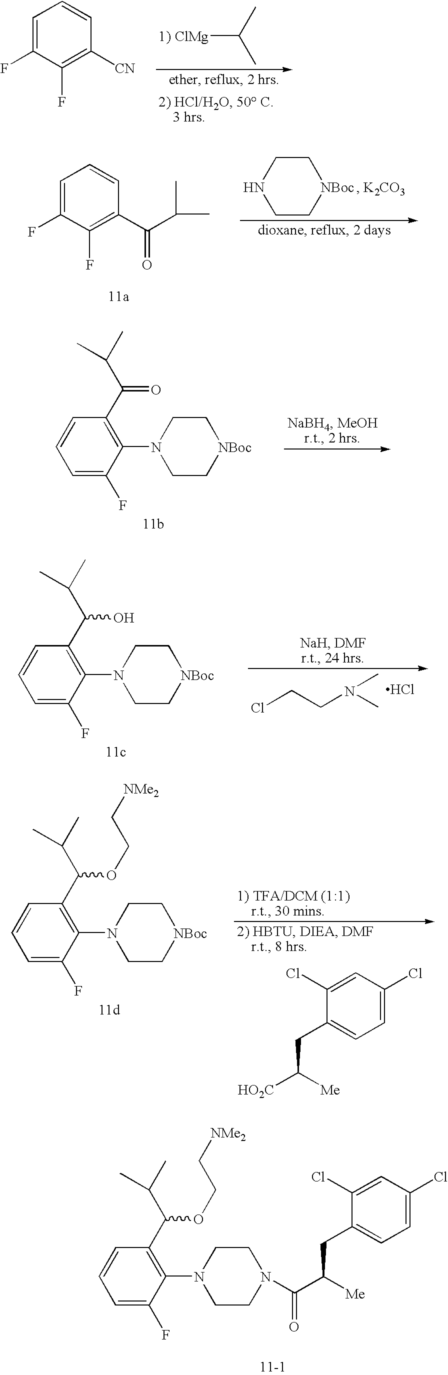

### Step 11A: Compound 11b

To a flame-dried flask, 2,3-difluorobenzonitirle (10 g, 72 mmol) was added along with dry ether (18 mL) and the mixture was allowed to stir at room temperature under nitrogen atmosphere. To the reaction flask, isopropyl Grignard reagent (72 mL, 2M in ether) was added slowly. The reaction mixture was then allowed to reflux (˜40° C.) for 2 hours. The reaction was then cooled to 0° C. and was quenched with a mixture of water (30 mL) and 2N HCl (60 mL). The biphasic reaction mixture stirred at 50° C. for 3 hours then cooled to room temperature. The organic layer was separated and the aqueous phase was extracted with ether (2×75 mL). The organic phases were combined and were washed with saturated NaHCO₃ solution (2×100 mL) followed by washing with saturated NaCl solution (100 mL). The organic layer was isolated, dried over anhydrous MgSO₄, filtered, and evaporated to dryness in vacuo. Difluorophenyl ketone 11a, was recovered in 86% yield (11.6 g, 63 mmol) after purification by column chromatography on silica using 30% hexanes/dichloromethane as the eluent (R_{f}=0.8). Compound 11a (11.6 g, 63 mmol) was dissolved in 1,4-dioxane (63 mL) along with potassium carbonate (17.5 g, 127 mmol) and I-boc-piperazine (11.8 g, 63 mmol). The reaction mixture was allowed to reflux for 2 days then cooled to room temperature and filtered to remove potassium carbonate. Solvent from the mother liquor was removed under vacuum and the residue was purified by column chromatography on silica using 10% ethyl acetate/hexanes as the eluent (R_{f}=0.3) to give 11b (15.8, 45 mmol, 71% yield). MS: calc. for C₁₉H₂₇FN₂O₃: 350.2; Found: 351.0 (M+H); retention time: 2.24 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 11B: Compound 11d

Fluorophenyl ketopiperazine 11b (5.6 g, 16 mmol) was dissolved in MeOH (64 mL) and sodium borohydride (0.97 g, 25.6 mmol) was added in several portions. The reaction mixture was allowed to stir at room temperature for 2 hours then quenched with 1N NaOH solution. Ether was added to the reaction mixture (150 mL) and the organic layer was separated. The aqueous phase was extracted with ether (2×100 mL) then the organic phases were combined, washed with saturated NaHCO₃ solution (2×150 mL) followed by washing with saturated NaCl solution (150 mL). The organic layer was isolated, dried over anhydrous MgSO₄, filtered, and evaporated to dryness in vacuo. The alcohol intermediate 11c was isolated in quantitative yield (5.7 g, 16 mmol) and a portion was used for the next step without any further purification. The alcohol intermediate 1c (3.5 g, 10 mmol) was added to DMF (100 mL) containing sodium hydride (1.2 g, 30 mmol, 60% dispersion in oil). The reaction mixture was allowed to stir at room temperature under nitrogen atmosphere for 1 hour then 2-dimethylaminoethyl chloride hydrochloride (2.2 g, 15 mmol) was added in one portion. The reaction was allowed to stir for 12 hours then additional sodium hydride (0.4 g, 10 mmol, 60% dispersion in oil) was added and stirring was continued for another 12 hours at room temperature. The reaction mixture was quenched with water then partitioned between water (250 mL) and ethyl acetate (250 mL). The organic layer was washed with water (2×200 mL), saturated NaHCO₃ solution (200 mL) followed by washing with saturated NaCl solution (200 mL). The organic layer was isolated, dried over anhydrous MgSO₄, filtered, and evaporated to dryness in vacuo. Compound 11d was recovered in 44% yield (1.86 g, 4.4 mmol) after purification by column chromatography on silica using 10% methanol/dichloromethane as the eluent (R_{f}=0.35). MS: calc. for C₂₃H₃₈FN₃O₃: 423.3; Found: 424.1 (M+H); retention time: 2.511 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 11C: Compound 11-1

2-Fluorophenyl ether 1d (85 mg, 0.2 mmol) was dissolved in 4 mL of (1:1) TFA/DCM and stirred at room temperature for 30 minutes. The reaction mixture was concentrated under a stream of nitrogen then diluted with dichloromethane (8 mL). The organic layer was washed with saturated NaHCO₃ solution (3×20 mL) followed by washing with saturated NaCl solution (20 mL). The organic layer was then isolated, dried over anhydrous MgSO₄, filtered, and evaporated to dryness in vacuo. The residue was dissolved in DMF (1 mL) along with HBTU (76 mg, 0.2 mmol) and DIEA (35 μl, 0.4 mmol). The reaction mixture was stirred at room temperature for 1 hour then dichlorophenyl propionic acid (47 mg, 0.2 mmol) was added and the reaction was stirred for an additional 7 hours. The reaction was diluted with ethyl acetate (4 mL), washed with saturated NaHCO₃ solution (3×10 mL) followed by washing with saturated NaCl solution (10 mL). The organic layer was then isolated, dried over anhydrous MgSO₄, filtered, and evaporated to dryness in vacuo. The crude product was purified by preparative HPLC to give compound 11-1 as the TFA salt in 44% yield. MS: calc. for C₂₈H₃₈Cl₂FN₃O₂: 537.2; Found: 538 (M+H); retention time: 5.475 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 11 were prepared.

TABLE 11

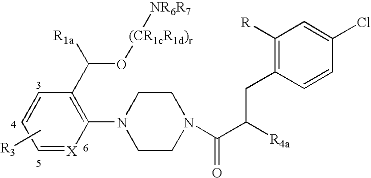

| | | —(CR_{1c}R_{1d})ᵣ— | | | | | | | |
|---|---|---|---|---|---|---|---|---|---|
| Cpd | X | NR₆R₇ | R₃ | R₁ₐ | R | R₄ₐ | [M + H]⁺ | MW | t_{R} |
| 11-1 | CH | —CH₂CH₂NMe₂ | 4- | i-Bu- | Me | Me | 528.3 | 528.18 | — |
| | | | Me | | | | | | |
| 11-2 | CR₃ | —CH₂CH₂NMe₂ | 6-F | i-Pr- | H | Me | 504.1 | 504.09 | 5.267 |
| 11-3 | CR₃ | —CH₂CH₂NMe₂ | 6-F | i-Pr- | F | Me | 522.1 | 522.08 | 5.322 |
| 11-4 | CR₃ | —CH₂CH₂CH₂NMe₂ | 6-F | i-Pr- | Cl | Me | 551.7 | 552.56 | 7.448 |
| 11-5 | CR₃ | —CH₂CH₂NMe₂ | 6-F | i-Pr- | H | 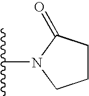 | 573.1 | 573.15 | 5.085 |
| 11-6 | CR₃ | —CH₂CH₂NMe₂ | 6-F | i-Pr- | Cl | 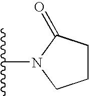 | 607.1 | 607.59 | 5.344 |
| 11-7 | CR₃ | —CH₂CH₂CH₂NMe₂ | 6-F | i-Pr- | Cl | 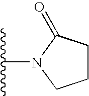 | 621.7 | 621.62 | 6.688 |

### Example 12

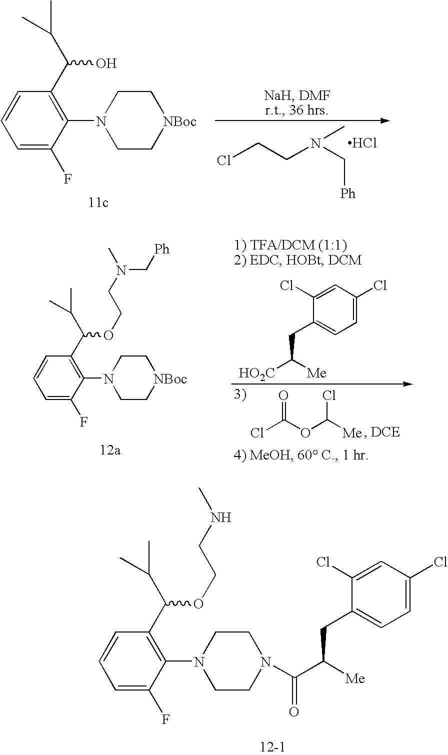

### Step 12A: Compound 12a

The 2-fluorophenyl alcohol 11c (453 mg, 1.3 mmol) was added to DMF (8 mL) containing sodium hydride (154 mg, 3.8 mmol, 60% dispersion in oil). The reaction mixture was allowed to stir at room temperature under nitrogen atmosphere for 1 hour then 2-benzyl-2-methylaminoethyl chloride hydrochloride (564 mg, 2.6 mmol) was added in one portion. The reaction was allowed to stir for 36 hours at room temperature. The reaction mixture was quenched with water then partitioned between water (20 mL) and ethyl acetate (20 mL). The organic layer was washed with water (2×20 mL), saturated NaHCO₃ solution (20 mL) followed by washing with saturated NaCl solution (20 mL). The organic layer was isolated, dried over anhydrous MgSO₄, filtered, and evaporated to dryness in vacuo. 2-Fluorophenyl aminoether 12a was recovered in 34% yield (0.22 g, 0.45 mmol) after purification by column chromatography on silica using 10% acetone/dichloromethane as the eluent (R_{f}=0.35). MS: calc. for C₂₉H₄₂FN₃O₃: 499.3; Found: 500 (M+H); retention time: 2.535 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 12B: Compound 12-1

2-Fluorophenyl aminoether 12a (0.22 g, 0.45 mmol) was dissolved in 6 mL of (1:1) TFA/DCM and stirred at room temperature for 1 hour. The reaction mixture was concentrated under a stream of nitrogen then diluted with dichloromethane (15 mL). The organic layer was washed with saturated NaHCO₃ solution (3×30 mL) followed by washing with saturated NaCl solution (30 mL). The organic layer was then isolated, dried over anhydrous MgSO₄, filtered, and evaporated to dryness in vacuo. The crude deprotected intermediate was recovered in quantitative yield and an aliquot was used for the next step without any further purification. The deprotected piperazine intermediate (90 mg, 0.23 mmol) was dissolved in DCM (1 mL) along with HOBt (30 mg, 0.23 mmol) and dichlorophenyl propionic acid (52 mg, 0.23 mmol). The reaction mixture was allowed to stir at room temperature for 10 minutes then EDC (43 mg, 0.23 mmol) was added. The mixture was then allowed to stir at room temperature for an additional 8 hours. After 8 hours, the reaction mixture was diluted with dichloromethane (5 mL) then washed with saturated NaHCO₃ (3×5 mL) and saturated NaCl (5 mL). The organic layer was collected, dried over anhydrous MgSO₄, filtered, and evaporated to dryness under vacuum. The residue was used without any further purification. The crude residue (138 mg, 0.23 mmol) was dissolved in 1,2-dichloroethane (2 mL) along with proton sponge (48 mg, 0.23 mmol) and cooled to 0° C. To the reaction mixture, ACE-Cl (73 μL, 0.68 mmol) was added slowly at 0° C. then the reaction was allowed to stir under nitrogen atmosphere at 75° C. for 3 hours. The reaction mixture was then concentrated under a stream of nitrogen and the resulting residue was dissolved in methanol (2 mL). The reaction mixture was allowed to stir at 65° C. for 1 hour then solvent was removed in vacuo. The crude product was purified by preparative HPLC. Compound 12-1 was recovered as the TFA salt in 8.6% yield. MS: calc. for C₂₇H₃₆Cl₂FN₃O₂: 523.2; Found: 523.7 (M+H); retention time: 7.155 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 12 were prepared.

TABLE 12

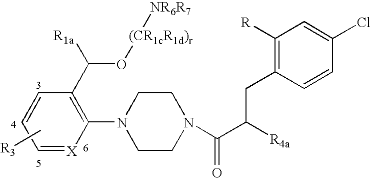

| Cpd | X | —(CR_{1c}R_{1d})ᵣ—NR₆R₇ | R₃ | R₁ₐ | R | R₄ₐ | [M + H]⁺ | MW | t_{R} |
|---|---|---|---|---|---|---|---|---|---|
| 12-1 | CR₃ | —CH₂CH₂CH₂NHMe | 6-F | i-Pr- | Cl | Me | 537.7 | 538.53 | 7.190 |
| 12-2 | CR₃ | —CH₂CH₂NHMe | 6-F | i-Pr- | Cl | Me | 523.7 | 524.5 | 7.155 |
| 12-3 | CR₃ | —CH₂CH₂CH₂NHMe | 6-F | i-Pr- | Cl | 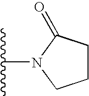 | 606.7 | 607.59 | 6.603 |
| 12-4 | CR₃ | —CH₂CH₂NHMe | 6-F | i-Pr- | Cl | 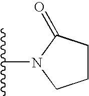 | 592.7 | 593.57 | 6.493 |

### Example 13

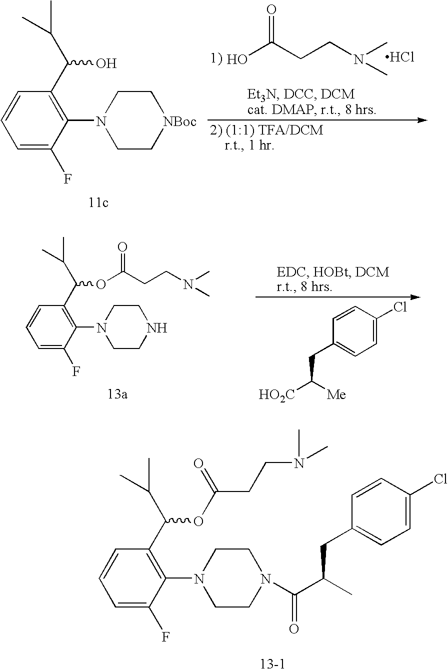

### Step 13A: Compound 13a

The 2-fluorophenyl alcohol 11c (352 mg, 1 mmol) was dissolved in dichloromethane (5 mL) along with 3-dimethylaminopropionic acid hydrochloride (154 mg, 1 mmol), triethylamine (141 μL, 1 mmol), DCC (206 mg, 1 mmol), and DMAP (12 mg, 0.1 mmol). The reaction mixture was allowed to stir at room temperature for 8 hours then the reaction mixture was diluted with dichloromethane (5 mL), washed with saturated NaHCO₃ (3×10 mL) and saturated NaCl (10 mL). The organic layer was collected, dried over anhydrous MgSO₄, filtered, and evaporated to dryness under vacuum. The residue was used for the next step without any further purification. The intermediate aminoester (0.42 g, 0.93 mmol) was dissolved in 4 mL of (1:1) TFA/DCM and stirred at room temperature for 1 hour. The reaction mixture was concentrated under a stream of nitrogen then diluted with dichloromethane (10 mL). The organic layer was washed with saturated NaHCO₃ solution (3×20 mL) followed by washing with saturated NaCl solution (20 mL). The organic layer was then isolated, dried over anhydrous MgSO₄, filtered, and evaporated to dryness in vacuo to give 13a which was used in the next step without further purification.

### Step 13B: Compound 13-1

The deprotected piperazine intermediate 13a (190 mg, 0.54 mmol) was dissolved in DCM (2 mL) along with HOBt (73 mg, 0.54 mmol) and chlorophenyl propionic acid (107 mg, 0.54 mmol). The reaction mixture was allowed to stir at room temperature for 10 minutes then EDC (104 mg, 0.54 mmol) was added. The reaction was then allowed to stir at room temperature for an additional 8 hours. After 8 hours, the reaction mixture was diluted with dichloromethane (5 mL) then washed with saturated NaHCO₃ (3×10 mL) and saturated NaCl (10 mL). The organic layer was collected, dried over anhydrous MgSO₄, filtered, and evaporated to dryness under vacuum. A small aliquot of the crude product was purified by preparative HPLC. Compound 13-1 was recovered as the TFA salt (44 mg). MS: calc. for C₂₉H₃₉ClFN₃O₃: 531.2; Found: 531.8 (M+H); retention time: 6.525 minutes; Method info: APCI positive ion scan 100-1000 Frag V=80; 95% 0.025% TFA/H₂O to 95% ACN/0.025% TFA over 13 min, 15.5 min run, ODS-AQ column.

### Example 14

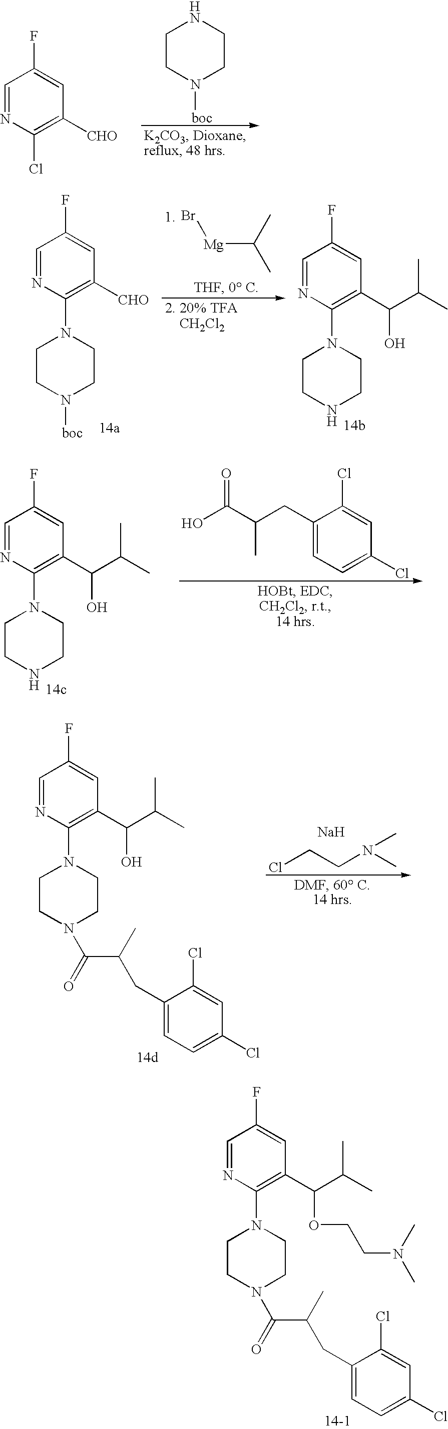

### Step 14A: Compound 14a

In a 250 mL flask, 2-chloro-5-fluoropyridine-3-carboxaldehyde (4.88 g, 31.0 mmol) was dissolved in dioxane (103 mL) along with Boc-piperazine (5.77 g, 31.0 mmol) and potassium carbonate (4.30 g, 31.0 mmol). The reaction was heated to reflux with stirring for 48 hours. The mixture was then diluted with ethyl acetate (100 mL) and washed with saturated NaHCO₃ solution (2×75 mL) and saturated NaCl solution (2×75 mL). The organic layer was collected, dried over anhydrous Na₂SO₄, and then filtered. Solvent was removed in vacuo and the residue was purified by column chromatography on silica using 9:1 hexane/ethyl acetate as the eluent to afford 3.0 g (31%) of the 14a as a yellow solid.

### Step 14B: Compound 14c

In a 100 mL round bottom flask, the aldehyde 14a (0.448 g, 1.45 mmol) was dissolved in THF (7 mL), placed under nitrogen, and then cooled to 0° C. Isopropyl Grignard (15% in THF, 11 mL, 1.60 mmol) was added dropwise while maintaining temperature below 0° C. After the addition, the reaction stirred for 20 minutes at 0° C. The reaction was slowly quenched with saturated NH₄Cl solution and then diluted with ethyl acetate (10 mL). The mixture was washed with saturated NaHCO₃ solution (5 mL) and then with saturated NaCl solution (5 mL). The organic layer was extracted, dried over anhydrous Na₂SO₄, filtered, and solvent removed in vacuo to afford the 14b as an oil in quantitative yield (0.55 g). LCMS (tᵣ, 2.736) M+H (354).

The Boc-piperazine 14b (0.552 g, 1.56 mmol) was dissolved in dichloromethane (15 mL), placed under nitrogen, and then treated with TFA (3.0 mL). The reaction stirred at room temperature for 30 minutes (reaction was monitored by TLC). The reaction was then neutralized with saturated NaHCO₃ solution and extracted with a 3:1 mixture of chloroform/isopropyl alcohol solution to give 0.260 g (65%) of 14c.

### Step 14C: Compound 14d

Compound 14c (0.284 g, 1.12 mmol) was dissolved in dichloromethane (3 mL) along with 2,4-dichloropropionic acid (0.260 g 1.12 g), and HOBt (0.182 g 1.35 mmol). The mixture was stirred for 20 minutes at room temperature. EDC (0.260 g, 1.35 mmol) was added and the mixture was stirred for 14 hours at room temperature. The reaction mixture was diluted with ethyl acetate (3 mL) and washed with saturated NaHCO₃ solution (2 mL). The organic layer was collected and solvent was removed under vacuum to afford 14d (0.366 g, 70%).

LCMS (tᵣ, 2.934) M+H (468)

### Step 14D: Compound 14-1

The hydroxyl intermediate 14d (0.048 g, 0.10 mmol) was dissolved in DMF (1.0 mL). NaH (60% in oil, 0.016 g, 0.40 mmol) was added and the reaction stirred at room temperature for 1 hour. Then, dimethylamino ethyl chloride (0.015 g, 0.10 mml) was added and the reaction mixture was heated to 60° C. for 14 hours. The reaction mixture was diluted with ethyl acetate (1 mL) and was quenched with H₂O (2 mL). The organic layer was collected and solvent was reduced under a stream of nitrogen. The crude product was re-suspended in MeOH (1 mL) and purified by prep HPLC to give 14-1 (0.009 g, 17%) as the TFA salt. LCMS (tᵣ, 5.456) M+H (539).

By the above procedures, the compounds of the following Table 14 were prepared.

TABLE 14

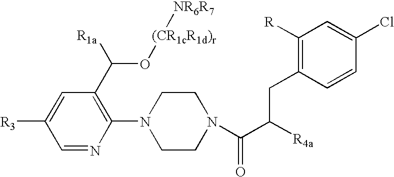

| | —(CR_{1c}R_{1d})ᵣ— | | | | | | | |
|---|---|---|---|---|---|---|---|---|
| Cpd | NR₆R₇ | R₃ | R₁ₐ | R | R₄ₐ | [M + H]⁺ | MW | t_{R} |
| 14-1 | —CH₂CH₂NMe₂ | F | i-Pr- | Cl | Me | 539.0 | 539.52 | 5.456 |
| 14-2 | —CH₂CH₂NEt₂ | F | i-Pr- | Cl | Me | 567.1 | 567.57 | 5.69 |
| 14-3 | —CH₂CH₂NEt₂ | F | i-Bu- | Cl | Me | 581.1 | 581.60 | 5.739 |
| 14-4 | 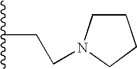 | F | Et- | Cl | Me | 551.0 | 551.53 | 5.470 |
| 14-5 | 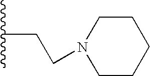 | F | Et- | Cl | Me | 565.0 | 565.56 | 5.471 |
| 14-6 | —CH₂CH₂NMe₂ | F | i-Bu- | Cl | Me | 552.0 | 553.55 | 5.520 |
| 14-7 | 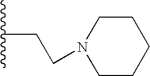 | F | Et- | Cl | Me | 565.0 | 565.56 | 5.471 |
| 14-8 | —CH₂CH₂NMe₂ | F | i-Pr- | Cl | 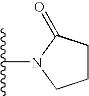 | 608.0 | 608.58 | 6.855 |
| 14-9 | —CH₂CH₂NMe₂ | F | i-Pr- | H | Me | 505.0 | 505.07 | 4.486 |
| 14-10 | —CH₂CH₂NMe₂ | H | i-Bu- | Cl | Me | 535.2 | 535.56 | 8.700 |
| 14-11 | —CH₂CH₂NEt₂ | H | i-Bu- | Cl | Me | 563.3 | 563.61 | 9.240 |
| 14-12 | 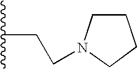 | H | i-Bu- | Cl | Me | 561.3 | 561.59 | 9.090 |
| 14-13 | 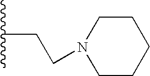 | H | i-Bu- | Cl | Me | 575.3 | 575.62 | 9.110 |

### Example 15

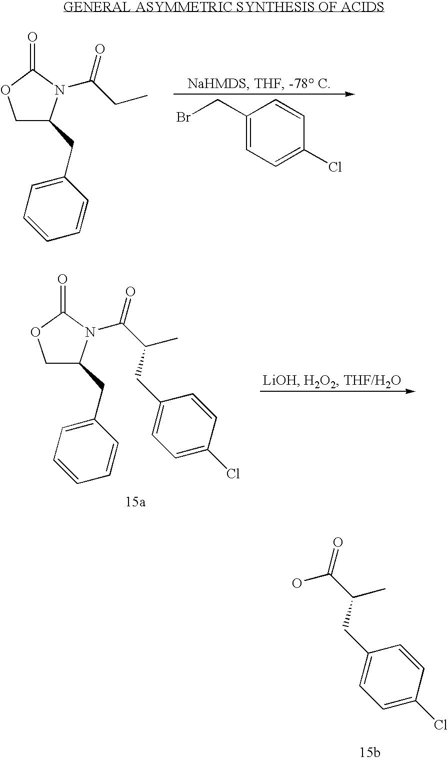

### Step 15A: Compound 15a

(S)-4-Benzyl-3-propionyl-oxazolidin-2-one (46.7 g, 200.0 mmol) was dissolved in THF (870 mL) under inert atmosphere (N₂). This was then cooled to −70° C. (dry ice/acetone) and treated with sodium hexamethyldisilazide-HMDS (110 mL of a 2.0M solution in THF, 220.0 mmol) in a dropwise fashion (addition lasted for 45 min.). The resulting mixture was stirred at −70° C. for 1 h. A solution of 4-chlorobenzyl bromide (53.4 g, 260.0 mmol) in THF (160 mL) was then added dropwise over 30 min. The resulting mixture was stirred at −70° C. for 6 h and then allowed to warm to room temperature overnight. The reaction was carefully quenched with H₂O (100 mL) and the solvent was removed in vacuo. The resulting slurry was suspended in H₂O (200 mL) and filtered. The solid was rinsed with EtOAc and air-dried to give 36.67 g (102.6 mmol, 51%) of 15a. A second crop was obtained from the filtrates after the organic layer was separated, washed with brine, dried (MgSO₄) and evaporated. The resulting brown solid was suspended in MeOH and filtered to give 17.22 g (48.2 mmol, 24%) of 15a. Only one diastereomer was observed by ¹H NMR and by single crystal X-ray analysis. ¹H NMR (CDCl₃-300 MHz) δ 1.18 (d, J=6.3 Hz, 3H,); 2.66-2.56 (m, 2H); 3.16-3.07 (m, 2H); 4.22-4.02 (m, 3H); 4.71-4.63 (m, 1H); 7.08-7.05 (m, 2H); 7.32-7.21 (m, 7H).

### Step 15B: Compound 15b

Oxazolidinone 15a (53.89 g, 150.7 mmol) was dissolved in a 4:1 v/v THF/H₂O mixture (750 mL) and cooled to 0° C. (ice/water bath). Hydrogen peroxide 50% (60 mL) was added slowly, followed by a solution of lithium hydroxide monohydrate (11.08 g, 263.7 mmol) in H₂O (380 mL). The resulting mixture was stirred at 0° C. for 1.5 h. Na₂SO₃.7H₂O (155.70 g, 618.0 mmol) dissolved in H₂O (250 mL) was added at 0° C., and the resulting mixture was allowed to warm to room temperature slowly. The volatiles were removed in vacuo and the aqueous residue was extracted with CH₂Cl₂ (2×300 mL). The aqueous layer was separated, made acidic w/2.0N HCl and then extracted with EtOAc (2×250 mL). The organics were washed with brine, dried over MgSO₄ and evaporated to give 15b as an oil that solidified upon standing. Yield=29.80 g (150.1 mmol, 99%). ¹H NMR (CDCl₃-300 MHz) δ 1.18 (d, J=6.3 Hz, 3H,); 2.80-2.62 (m, 2H); 3.02 (dd, J₁=6.0 Hz, J₂=12.6 Hz, 1H); 7.12 (d, J=8.7 Hz, 2H); 7.26 (dd, J=8.7 Hz, 2H). [α]_{D}=−27.930 (c=8.685 mg/cc, MeOH).

### Example 16

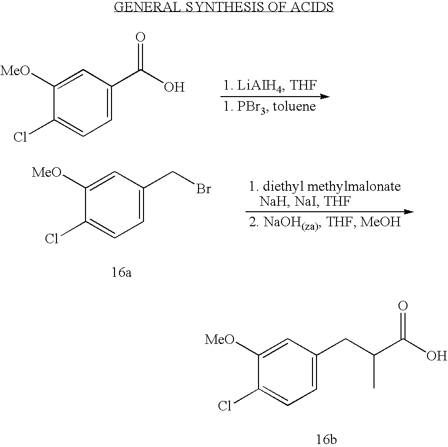

### Step 16A: Compound 16a

To a solution of 4-chloro-3-methoxy-benzoic acid (2.04 g, 11.0 mmol) in 100 mL THF at 0° C. was added LiAlH₄ (417 mg, 11.0 mmol). The mixture was stirred at the same temperature for 2 h and quenched with sat. NH₄Cl solution. The aqueous layer was extracted with EtOAc (100 mL, twice) and the combined organic layers were washed with brine, dried over Na₂SO₄, filtered and concentrated to give 1.70 g of material which was dissolved in 10 mL toluene. To the solution was added PBr₃ (0.33 mL, 3.45 mmol). The reaction was stirred for 2 h at room temperature and diluted with Et₂O, washed with H₂O twice, then brine. The solution was dried over Na₂SO₄, filtered, and concentrated to give 16a (2.16 g, 9.2 mmol) as an oil which solidified upon standing.

### Step 16B: Compound 16b

Diethyl methylmalonate (1.37 mL, 8.7 mmol) was added dropwise to a suspension of NaH (60% in oil, 348 mg, 8.7 mol) in THF (20 mL). The generation of H₂ was visible. The mixture was stirred for 0.5 h at room temperature and a solution of 16a (2.16 g, 9.2 mmol) in 10 mL THF and NaI (catalytic amount) was added. The reaction mixture was stirred overnight and then was worked up by adding H₂O and extracting with Et₂O twice. The combined organic layers were washed with brine, dried over Na₂SO₄, and concentrated to give a clear oil which was dissolved in 5 mL THF. MeOH (5 mL) and NaOH (2N, 20 mL) were added to the solution and the mixture was heated at 80° C. for 4 h. After cooling, the mixture was extracted with Et₂O and the aqueous solution was acidified using 6 N HCl. The solution was extracted with EtOAc three times. The organic layers were combined and washed with brine, dried over Na₂SO₄, filtered, and concentrated. The crude material was trituated with hexane to afford 16b as a white solid (885 mg, 3.86 mmol).

### Example 17

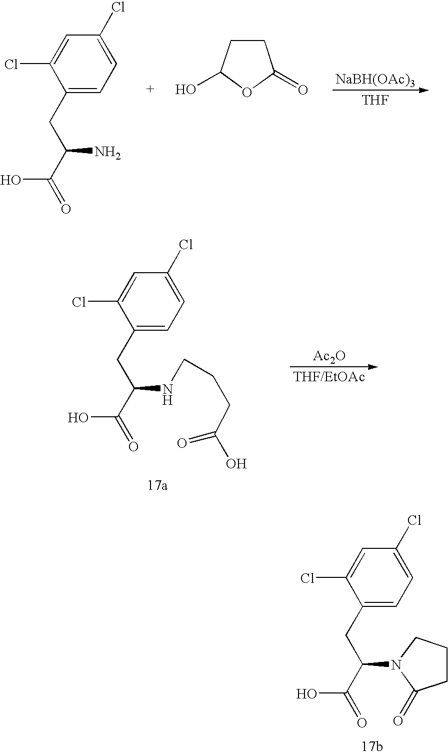

### Step 17A: Compound 17a

To 35.11 g (150 mmol) of D-2,4-dichlorophenylalanine in 700 mL of THF was added 150 mL (225 mmol) of a 15% succinic semialdehyde in water solution. The mixture was stirred for 1 hour at room temperature. Sodium triacetoxyborohydride (63.6 g, 300 mmol) was added in three portions and the mixture stirred for 10 hours. The solution was carefully decanted to leave solid 17a. Acetic anhydride (100 mL), 250 mL of THF and 250 mL of EtOAc were added and the reaction mixture was stirred vigorously. The mixture was heated to ˜50° C. for two hours. The THF and EtOAc were removed in vacuo and the residue poured slowly into 1 L of vigorously stirred water. White precipitate was formed, filtered, and dried to give 17b (31.5 g) as a white solid.

### Example 18

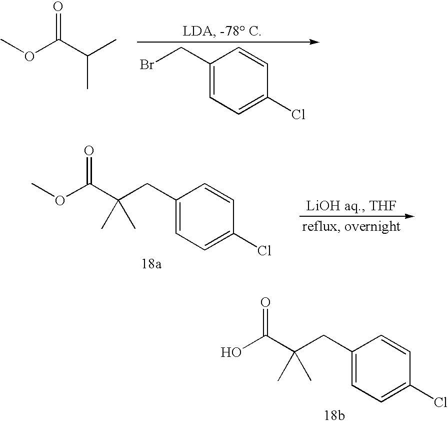

### Step 18A: Compound 18a

A solution of methyl isobutyrate (1.15 mL, 10.0 mmol) in THF (3 mL) was added dropwise to a stirring solution of lithium diisopropylamide (LDA) (5 mL of a 2.0 M solution in THF, 10.0 mmol) in THF (8 mL) at −78° C. (dry ice/acetone bath) under N₂. After 30 min., a solution of 4-chlorobenzyl bromide (2.26 g, 11.0 mmol) in THF (3 mL) was added, and the resulting mixture was stirred at −78° C. for 6 h. The reaction was quenched carefully with H₂O and slowly warmed to room temperature. The reaction mixture was diluted with EtOAc (100 mL), then washed with 5% aqueous HCl and brine. The organics were dried over anhydrous MgSO₄, filtered and concentrated in vacuo to give 3-(4-chloro-phenyl)-2,2-dimethyl-propionic acid methyl ester 18a as an orange oil (2.18 g, 9.6 mmol, 96%).

### Step 18B: Compound 18b

3-(4-Chlorophenyl)-2,2-dimethyl-propionic acid methyl ester (2.18 g, 9.6 mmol) was dissolved in THF (30 mL) and treated with LiOH (15 mL of a 2.0 M solution in H₂O). The resulting mixture was refluxed overnight. After cooling, the reaction was diluted with H₂O and extracted with EtOAc. The layers were separated and the aqueous layer was acidified to pH=1 with 2 N HCl. The aqueous layer was extracted with EtOAc (100 mL) and this ethyl acetate layer was washed with brine, dried over anhydrous MgSO₄, filtered and concentrated in vacuo to give 3-(4-chlorophenyl)-2,2-dimethyl-propionic acid 18b as a pale yellow oil (1.23 g, 5.8 mmol, 60%).

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

It will be appreciated that, although specific embodiments of the invention have been described herein for purposes of illustration, various modifications may be made without departing from the spirit and scope of the invention. Accordingly, the invention is not limited except as by the appended claims.

1. A compound having the following structure: 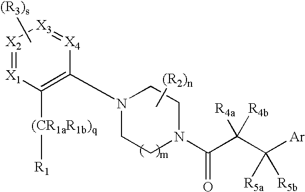
or a pharmaceutically acceptable salt, ester, solvate, stereoisomer, or prodrug thereof,
wherein:
Ar is aryl, substituted aryl, heteroaryl, or substituted heteroaryl;
X₁, X₂, X₃ and X₄ are independently CH or N, with the proviso that no more than two of X₁, X₂, X₃ and X₄ are N, and with the further proviso that when two of X₁, X₂, X₃ and X₄ are N either X₁ and X₃ are both N or X₂ and X₄ are both N;
R₁ is (Y₁—Y₂)—NR₆R₇, —NR₈C(═O)R₉, —NR₈S(O)ₚR₁₀, —NR₈C(═O)OR₁₁, imidazolyl, triazolyl, oxazolyl, or thiazolyl;
Y₁ is —O—, —S— —NR₈—, —C(═O)—, —C(═O)O—, —OC(═O)—, —NR₈C(═O)O—, —NR₈C(═O)—, —C(═O)NR₈—, —NR₈S(═O)ₚ—, —S(═O)ₚ—, —S(═O)ₚNR₈—, or —NR₈C(═O)NR₈—;
Y₂ is —(CR_{1c}R_{1d})ᵣ—;
R₁ₐ, R_{1b}, R_{1c}, and R_{1d} are at each occurrence the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
or R₁ₐ and R_{1b} taken together with the carbon atom to which they are attached form a homocycle or substituted homocycle;
or R_{1c} and R_{1d} taken together with the carbon atom to which they are attached form a homocycle or substituted homocycle;
or R_{1c} and R₆ taken together form a heterocycle or substituted heterocycle;
R₂ is at each occurrence the same or different and independently alkyl or substituted alkyl, and where n is 0, 1, 2, 3 or 4 and represents the number of R₂ substituents;
R₃ is at each occurrence the same or different and independently hydroxy, halogen, cyano, nitro, alkyl, haloalkyl, substituted alkyl, aryl, substituted aryl, heterocycle, or substituted heterocycle, and where s is 0, 1 or 2 and represents the number of R₃ substituents;
R₄ₐ and R₅ₐ are the same or different and independently hydrogen, methyl, hydroxy, halogen, dialkylamino, substituted dialkylamino, heterocycle, or substituted heterocycle;
R_{4b} band R_{5b} are the same or different and independently hydrogen or methyl;
R₆ and R₇ are the same or different and independently are hydrogen, alkyl, substituted alkyl, aryl, substituted aryl, arylalkyl, substituted arylalkyl, heterocycle, substituted heterocycle, heterocyclealkyl, or substituted heterocyclealkyl,
or R₆ and R₇ taken together with the nitrogen atom to which they are attached form a heterocyclic ring or a substituted heterocyclic ring;
R₈ is at each occurrence the same or different and independently hydrogen, alkyl, substituted alkyl, —C(═O)R₉, or —SO₂R₁₀;
R₉, R₁₀ and R₁₁ are are each occurrence the same or different and independently hydrogen, alkyl, substituted alkyl, aryl, or substituted aryl;
m and p are the same or different and independently 0, 1 or 2; and
q and r are the same or different and independently 0, 1, 2, 3 or 4.

2. The compound of claim 1 wherein X₁, X₂, X₃, and X₄ are all CR₃ or CH.

3. The compound of claim 1 wherein one of X₁, X₂, X₃, and X₄ is N.

4. The compound of claim 1 wherein X₁ and X₃ are both N or X₂ and X₄ are both N.

5. The compound of claim 1 wherein R₁ is —Y₁—Y₂—NR₆R₇.

6. The compound of claim 5 wherein Y₁ is —O—, —S— —NR₈—, —C(═O)—, —C(═O)O—, or —OC(═O)—.

7. The compound of claim 5 wherein Y, is —NR₈C(═O)O—, —NR₈C(═O)—, —C(═O)NR₈—, —NR₈S(═O)ₚ—, —S(═O)ₚ—, —S(═O)ₚNR₈—, or —NR₈C(═O)NR₈—.

8. The compound of claim 1 wherein R₁ is —NR₈C(═O)R₉, —NR₈S(O)ₚR₁₀ or —NR₈C(═O)OR₁₁

9. The compound of claim 1 wherein R₁ is imidazolyl, triazolyl, oxazolyl, or thiazolyl.

10. The compound of claim 1 wherein Ar is aryl or substituted aryl.

11. The compound of claim 1 wherein Ar is heteroaryl or substituted heteroaryl.

12. The compound of claim 1 wherein R₆ and R₇ are the same or different and independently are hydrogen, alkyl, substituted alkyl, aryl, substituted aryl, arylalkyl, substituted arylalkyl, heterocycle, substituted heterocycle, heterocyclealkyl, or substituted heterocyclealkyl.

13. A pharmaceutical composition comprising a compound according to any one of claims 1 to 12 and a pharmaceutically acceptable carrier or diluent.

14. A method for treating a patient having a disorder associated with the activity of a melanocortin receptor, comprising administering to the patient a pharmaceutical composition comprising a pharmaceutically effective amount of a compound of claim 1 and a pharmaceutically acceptable carrier or diluent.

15. The method of claim 14 wherein the melanocortin receptor is melanocortin 3 receptor.

16. The method of claim 14 where the melanocortin receptor is melanocortin 4 receptor.

17. The method of claim 14 wherein the compound is an antagonist of the melanocortin receptor.

18. The method of claim 14 wherein the compound is an agonist of the melanocortin receptor.

19. The method of claim 14 wherein the disorder is an eating disorder.

20. The method of claim 19 wherein the eating disorder is cachexia.

21. The method of claim 14 wherein the disorder is a sexual disfunction.

22. The method of claim 21 where the sexual disfunction is erectile disfunction.

23. The method of claim 14 wherein the disorder is a skin disorder.

24. The method of claim 14 where the disorder is chronic pain.

25. The method of claim 14 where the disorder is anxiety or depression.

26. The method of claim 14 wherein the disorder is obesity.
